(19) United States
(12) Patent Application Publication
(10) Pub. No.: US 2018/0000968 A1
(43) Pub. Date: Jan. 4, 2018

(54) Poly(Beta-Amino Ester)s With Additives for Drug Delivery

(71) Applicant: Sagetis Biotech, S.L., Barcelona (ES)

(72) Inventors: Sejin OH, Barcelona (ES); Salvador BORROS GOMEZ, Barcelona (ES)

(21) Appl. No.: 15/545,421

(22) PCT Filed: Jan. 21, 2016
(86) PCT No.: PCT/IB2016/050299

§ 371 Date: Jul. 21, 2017

(30) Foreign Application Priority Data
Jan. 21, 2015 (GB) 1501025.9

(51) Int. Cl. A61K 48/00; A61K 9/51; C08G 73/02

(52) U.S. Cl. CPC A61K 48/0008; C08G 73/02; A61K 48/0091; A61K 9/5123; A61K 9/5161; A61K 9/5146; A61K 9/5192

(57) ABSTRACT

Disclosed are nanoparticles comprising an end-modified poly(β-amino ester) and an additive that is a sugar or sugar derivative, such as a sugar, a sugar alcohol or chitosan. The nanoparticles may be used in any field where polymers have been found useful, including in medical fields, particularly in drug delivery. The polymers are useful in delivering a polynucleotide such as DNA, RNA or siRNA, a small molecule or a protein. Also disclosed are compositions comprising said nanoparticles and an active agent, methods for preparing said nanoparticles, said nanoparticles and compositions for use in medicine, and in vitro methods using said nanoparticles and compositions.

The invention relates to nanoparticles that are suitable for use in delivery of active agents comprising poly(β-amino ester)s (PBAEs) and additives that are sugars or sugar derivatives. The invention also pertains to compositions comprising these nanoparticles and methods for their production.

The lack of safe and efficient vectors to deliver polynucleotides such as DNA and RNA remains the principal handicap for the success of gene therapy (Luo, D. & Saltzman, W. M. Synthetic DNA delivery systems. Nature Biotech. 18, 33-37 (2000); Kamimura K. et al, Advances in Gene Delivery Systems. Pharmaceut. Med. 25, 293-306 (2011); Miele E. et al, Nanoparticle-based delivery of small interfering RNA: challenges for cancer therapy. Int. J. Nanomedicine 7, 3637-3657 (2012)). The majority of protocols for polynucleotide delivery employ viral vectors, which are highly efficient delivery systems. However, viral vectors have certain disadvantages, including safety risk, limited capacity to carry polynucleotides and high cost of large-scale production. Non-viral vectors offer potential advantages, including high packing capacity, ease of production, low toxicity and immunogenicity, but are less efficient than viral vectors (Mintzer, M. A. & Simanek, E. E. Nonviral vectors for gene delivery. Chem. Rev. 109, 259-302 (2009)).

Biodegradable PBAEs have been described as potential non-viral polynucleotide delivery vectors capable of condensing both DNA and RNA into discrete nanometric particles (Green, J. J. et al. Acc. Chem Res. 41, 749-759 (2008)). Chemical modification at the termini of PBAEs with primary amines has been shown to produce higher transfection efficacy than commercial transfection agents such as Lipofectamine 2000, Fugene and polyethylenimine (PEI) (Zugates, G. T. et al. Bioconjugate Chem. 18, 1887-1896 (2007); Green, J. J. et al. Nano letters 8, 3126-3130 (2008); WO02/31025A2).

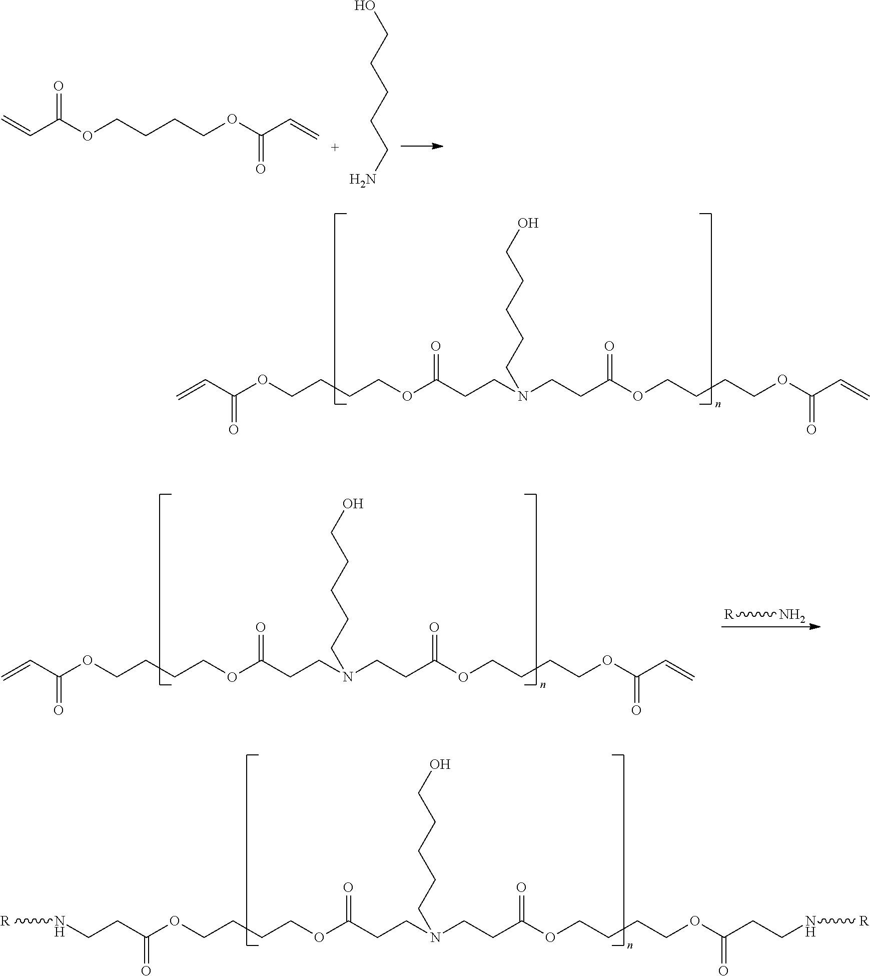

PBAEs chemically modified at one or both termini with oligonucleotides have been shown to be more biocompatible to cells, to result in high gene expression levels and to be able to deliver polynucleotides directly to cells in vitro without the need for ligand-mediated mechanisms (N. Segovia et al. Acta Biomateralia 10, 2147-2158 (2014); WO2014/136100).

Despite the development of PBAEs for transfection of polynucleotides into cells, their use has not been generally commercialized due to a propensity for rapid aggregation or disassembly in the phosphate-buffered saline (pH 7.4).

Chitosan has been studied as a candidate for nucleotide delivery. In recent years, chitosan-based carriers have been of interest as non-viral vectors that can provide not only a safe delivery system for gene materials (Mao 2010) but also stable nanoparticles.

Freeze-drying is a widely used process for drying and improving the stability of various pharmaceutical products. Various excipients are used as cryoprotectants and/or lyoprotectants and to increase stability on storage. Sugars are popular cyoprotectants for freeze-drying nanoparticles, for example, trehalose, sucrose and mannitol, which is of current interest as a drug delivery system for gene materials including DNA and/or preventing aggregation. Recently, complexes coated with poly(trehalose), obtained by a synthetic procedure of reversible addition-fragmentation chain transfer (RAFT) polymerization, were shown to promote stabilization and effective gene delivery (Sizovs et al. J. Am. Chem. Soc. 135, 15417-15424 (2013).

A continuing need exists for improved nanoparticles that are stable, non-toxic, biodegradable and biocompatible, that can be used to transfect polynucleotides efficiently and that can be prepared economically. Such nanoparticles would be useful in the packaging and delivery of DNA and RNA in gene therapy and for the packaging and delivery of other diagnostic, therapeutic and prophylactic agents.

In addition, there is a need for nanoparticles that can be used to efficiently transfect short polynucleotides, particularly siRNA and microRNA (miRNA), which have poor stability in circulation. Existing polymeric polynucleotide delivery vectors cannot encapsulate siRNA and miRNA with high loading owing to the relatively short length of these sequences. In addition, many existing polymeric delivery vectors for siRNA and miRNA are cytotoxic.

The present invention provides novel nanoparticles comprising end-modified PBAEs and a sugar or sugar alcohol. The present invention further provides novel nanoparticles comprising end-modified PBAEs and chitosan. The nanoparticles of the present invention are useful in a variety of medical applications including drug delivery, particularly in the delivery of polynucleotides; tissue engineering and biomaterials. The present invention is particularly directed to medical applications of these nanoparticles. The invention also provides methods of preparing the nanoparticles.

The inventors have found that the polyester nature of the end-modified PBAEs and the biological derivation of the chitosan, sugar or sugar alcohol provides an attractive biocompatible profile with high biodegradability and reduced toxicity. These nanoparticles have applications as non-viral polynucleotide delivery vectors in the treatment of many diseases such as cancer, monogenic diseases, vascular disease and infectious diseases. Another application of these nanoparticles is in vitro research as a tool to investigate gene function or regulation within a cellular and physiological context.

In a first aspect, the invention provides a nanoparticle comprising an end-modified poly(β-amino ester) and 1 to 35 weight percent of sugar or a sugar alcohol.

In a second aspect, the invention provides a nanoparticle comprising an end-modified poly(β-amino ester) wherein the nanoparticle has a coating comprising sugar or a sugar alcohol at 1 to 35 weight percent.

In a third aspect, the invention provides a nanoparticle comprising an end-modified poly(β-amino ester) and 0.15 to 3.0 weight percent chitosan or a pharmaceutically acceptable salt thereof.

Poly(β-amino ester)s may be formed from the reaction of di(acrylate ester) monomers with amino monomers, resulting in polymers having terminal acrylate groups that may be functionalized with end modifications.

Suitably, each end modification of the end-modified poly((3-amino ester) is independently selected from an oligopeptide and R_{y}; wherein R_{y} is selected from the group consisting of hydrogen, halogen, alkyl, cycloalkyl, alkenyl, cycloalkenyl, heteroalkyl, heterocycloalkyl, acyl, aryl or heteroaryl.

The end-modified poly(β-amino ester) is suitably a polymer of Formula I:

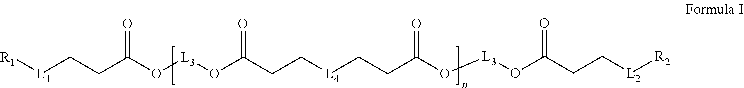

wherein
L₁ and L₂ are independently selected from the group consisting of:

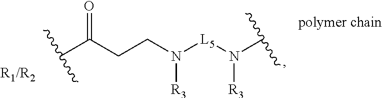

O, S, NRₓ and a bond; wherein Rₓ is independently selected from the group consisting of hydrogen, halogen, alkyl, cycloalkyl, alkenyl, cycloalkenyl, heteroalkyl, heterocycloalkyl, acyl, aryl or heteroaryl; L₃ is independently selected from the group consisting of alkylene, alkenylene, heteroalkylene, heteroalkenylene, arylene, heteroarylene and

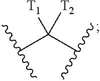

wherein T₁ is

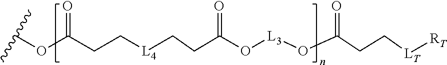

and
T₂ is selected from H, alkyl or

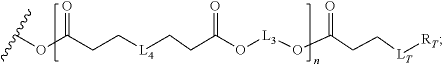

wherein L_{T} is independently selected from the group consisting of:

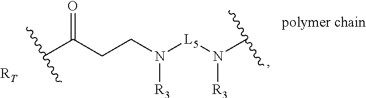

O, S, NRₓ and a bond; wherein Rₓ is independently selected from the group consisting of hydrogen, halogen, alkyl, cycloalkyl, alkenyl, cycloalkenyl, heteroalkyl, heterocycloalkyl, acyl, aryl or heteroaryl;
L₄ is independently selected from the group consisting of

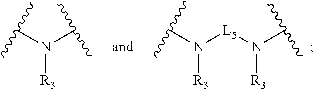

L₅ is independently selected from the group consisting of alkylene, alkenylene, heteroalkylene, heteroalkenylene, arylene or heteroarylene;
R₁, R₂ and R_{T}, where present, are independently selected from an oligopeptide and R_{y};
wherein R_{y} is selected from the group consisting of hydrogen, halogen, alkyl, cycloalkyl, alkenyl, cycloalkenyl, heteroalkyl, heterocycloalkyl, acyl, aryl or heteroaryl;
each R₃ is independently selected from the group consisting of hydrogen, halogen, alkyl, cycloalkyl, alkenyl, cycloalkenyl, heteroalkyl, heterocycloalkyl, acyl, aryl or heteroaryl; and
n is an integer from 5 to 10,000;
or a pharmaceutically acceptable salt thereof. In the polymer of formula I, R₁, R₂ and R_{T}, where present, are the end modifications.

Suitably, L₃ is independently selected from the group consisting of alkylene, alkenylene, heteroalkylene, heteroalkenylene, arylene and heteroarylene.

The present invention thus provides nanoparticles comprising end-modified PBAEs and a specific relative amount of a sugar, sugar alcohol or chitosan. These nanoparticles are particularly resistant to degradation and agglomeration whilst maintaining high transfection levels. They are therefore superior delivery agents. It is speculated that in nanoparticles according to the first aspect the sugar or sugar alcohol is either partially polymerized with the PBAE or is interspersed with the PBAE and thus stabilizes the resulting nanoparticles.

The nanoparticles have biodegradable groups capable of improving the delivery of polynucleotides to cells and have shown high transfection efficacy in vitro compared with chitosan alone and with commercial transfection agents.

The polymers of Formula I may be prepared by the reaction of diacrylate monomers of Formula II with substituted amines of formula L₄H₂ to form an acrylate terminated intermediate, Formula III.

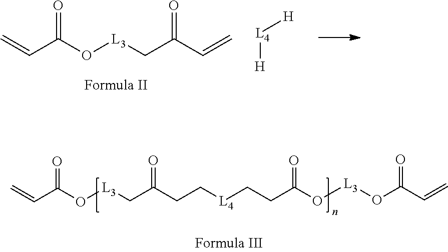

Groups R₁L₁ and R₂L₂ may then be added by reaction with a terminal acrylate group to form a polymer of Formula I.

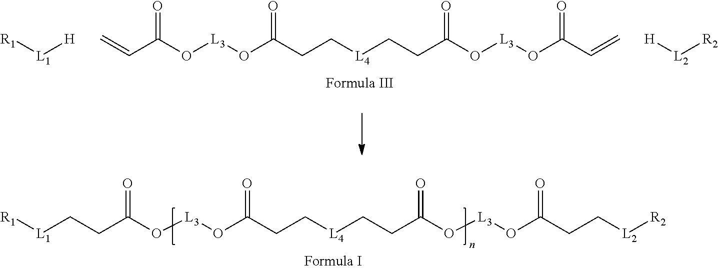

The first aspect of the invention is further directed to a method for preparing nanoparticles comprising the steps of (i) preparing an end-modified poly(β-amino ester) in the presence of a sugar or sugar alcohol and (ii) preparing nanoparticles from the product of step (i). Suitably, the sugar or sugar alcohol is present at a weight percent described herein relative to the weight of the end-modified poly(β-amino ester). Suitably, in step (i) the sugar or sugar alcohol is present in solution. For example, nanoparticles according to the first aspect of the invention may be prepared by (i) reacting the acrylate terminated intermediate of Formula III with compounds of formulae R₁L₁H and R₂L₂H in the presence of a sugar or sugar alcohol at the required weight percent and (ii) preparing nanoparticles from the product of step (i). Optionally, step (i) and step (ii) occur concurrently. Optionally, step (ii) is carried out in the presence of an active agent, which may be present in solution, or the method further comprises step (iii), comprising contacting the nanoparticles with an active agent, which may be present in solution.

Nanoparticles according to the first aspect of the invention may be prepared by a method according to the first aspect of the invention.

The second aspect of the invention is further directed to a method for preparing nanoparticles comprising the steps of (i) preparing nanoparticles from an end-modified poly(β-amino ester) and (ii) contacting the nanoparticles with a sugar or sugar alcohol. Suitably, the sugar or sugar alcohol is present at a weight percent described herein relative to the weight of end-modified poly(β-amino ester). Suitably, in step (ii) the sugar or sugar alcohol is present in solution. Optionally, step (ii) is carried out in the presence of an active agent, which may be present in solution. Suitably, the end-modified poly(β-amino ester) is a polymer according to Formula I, as described herein. Optionally, step (i) is carried out in the presence of an active agent, which may be present in solution, or the method further comprises, either between steps (i) and (ii) or after step (ii), a step comprising contacting the nanoparticles with an active agent, which may be present in solution. Suitably, step (i) is carried out in the presence of an active agent, which may be present in solution

Nanoparticles according to the second aspect of the invention may be prepared by a method according to the second aspect of the invention.

The third aspect of the invention is further directed to a method for preparing nanoparticles comprising the steps of (i) mixing an end-modified poly(β-amino ester) and with chitosan and (ii) preparing nanoparticles from the product of step (i). Suitably, the chitosan is present at a weight percent described herein relative to the weight of end-modified poly(β-amino ester). Suitably, in step (i) the chitosan is present in solution. Optionally, step (ii) is carried out in the presence of an active agent, which may be present in solution, or the method further comprises a step (iii) comprising contacting the nanoparticles with an active agent, which may be present in solution. Suitably, the end-modified poly(β-amino ester) is a polymer according to Formula I, as described herein.

Nanoparticles according to the third aspect of the invention may be prepared by a method according to the third aspect of the invention.

In polymers according to Formula I, each L₁, L₂ and L₁- is selected to facilitate coupling of the end-modifying groups R₁ R₂, and R_{T} to the PBAE polymer. Each L₁, L₂ and L_{T} may be a bond, for example where the end-modifying group is an oligopeptide that comprises a terminal cysteine residue.

In polymers according to Formula I, Rₓ may be independently selected from the group consisting of hydrogen, alkyl, cycloalkyl, alkenyl, cycloalkenyl, heteroalkyl and heterocycloalkyl, for example, from the group consisting of hydrogen, alkyl and cycloalkyl.

According to the present invention, an “oligopeptide” comprises a string of at least three amino acids linked together by peptide bonds. Such peptides preferably contain only natural amino acids, although non-natural amino acids (i.e., compounds that do not occur in nature but that can be incorporated into a polypeptide chain) and/or amino acid analogues as are known in the art may alternatively be employed. Also, one or more of the amino acids in such peptides may be modified, for example, by the addition of a chemical entity such as a carbohydrate group, a phosphate group, a farnesyl group, an isofarnesyl group, a fatty acid group, or a linker for conjugation, functionalization, or other modification, etc. The oligopeptides in the polymers of Formula I typically comprise from 3 to 20 amino acid residues, more preferably from 3 to 10 amino acid residues, more preferably from 3 to 6 amino acid residues. Alternatively, the oligopeptides in the polymers of Formula I may comprise from 4 to 20 amino acid residues, more preferably from 4 to 10 amino acid residues, more preferably from 4 to 6 amino acid residues.

Suitably, at least one of the end-modifying groups is an oligopeptide. For example, where nanoparticles comprise polymers of formula I, at least one of R₁ and R₂ is suitably an oligopeptide.

Suitably, the or each oligopeptide has a net positive charge at pH7. The or each oligopeptide may comprise naturally occurring amino acids that are positively charged at pH7, that is, lysine, arginine and histidine. For example, the or each oligopeptide may be selected from the group consisting of polylysine, polyarginine or polyhistidine, each of which may be terminated with cysteine.

The or each oligopeptide is suitably a compound of Formula IV:

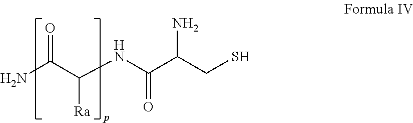

wherein p is an integer from 2 to 19, typically from 3 to 9 or from 3 to 5, and wherein Rₐ is selected at each occurrence from the group consisting of H₂NC(═NH)—NH(CH₂)₃—, H₂N(CH₂)₄— or (1H-imidazol-4-yl)-CH₂—.

Where the or each oligopeptide is a compound of Formula IV, the terminal cysteine residue provides a means of coupling the or each oligopeptide to the acrylate terminated intermediate. For example, the L₁, L₂ and/or L_{T} linking the or each oligopeptide to the polymer of formula I is a bond and, Formula III. The thiol functionality provides faster, more efficient and more easily controlled addition to the double bond. By contrast, where the or each oligopeptide is terminated in an amine functionality for coupling, an excess of this compound is required in the coupling step.

Suitably, the or each oligopeptide has a net negative charge at pH7. The or each oligopeptide may comprise naturally occurring amino acids that are negatively charged at pH7, that is, aspartic acid and glutamic acid. For example, the or each oligopeptide may be selected from the group consisting of polyaspartic acid and polyglutamic acid, each of which may be terminated with cysteine. For example, the or each oligopeptide may be a compound of Formula IV wherein p is an integer from 2 to 19, typically from 3 to 9 or from 3 to 5, and wherein Rₐ is HO₂C(CH₂)₂— or HO₂C—CH₂—. For example, the L₁, L₂ and/or L_{T} linking the or each oligopeptide to the polymer is a bond as the terminal cysteine residue provides a means of coupling the or each oligopeptide to the acrylate terminated intermediate, formula IV.

Alternatively, the or each oligopeptide may comprise a mixture of naturally occurring amino acids that are negatively charged at pH7 and naturally occurring amino acids that are positively charged at pH7.

Suitably, the or each oligopeptide is hydrophobic. The or each oligopeptide may comprise naturally occurring amino acids that are hydrophobic such as valine, leucine, isoleucine, methionine, tryptophan, phenylalanine, cysteine, tyrosine and alanine; in particular, the or each oligopeptide may comprise valine, leucine, isoleucine, methionine, tryptophan and phenylalanine.

Suitably, the or each oligopeptide is hydrophilic. The or each oligopeptide may comprise naturally occurring amino acids that are hydrophilic such as serine, threonine, cysteine, asparagine and glutamine, and may further comprise naturally occurring amino acids that are charged at pH7.

In the polymer of formula I both R₁ and R₂ may be oligopeptides or one of R₁ and R₂ may be an oligopeptide and the other of R₁ and R₂ may be R_{y}.

Where one of the end modifications is R_{y}, R_{y} is preferably selected from the group consisting of hydrogen, —(CH₂)ₘNH₂, —(CH₂)ₘNHMe, —(CH₂)ₘOH, —(CH₂)ₘCH₃, —(CH₂)₂(OCH₂CH₂)ₘNH₂, —(CH₂)₂(OCH₂CH₂)ₘOH and —(CH₂)₂(OCH₂CH₂)ₘCH₃ wherein m is an integer from 1 to 20, for example from 1 to 5. Preferably, R_{y} is selected from the group consisting of —(CH₂)ₘNH₂, —(CH₂)ₘNHMe and —(CH₂)₂(OCH₂CH₂)ₘNH₂. Preferably, when L₁ is NH or NRₓ, and one of R₁ and R₂ is R_{y}, then R_{y} is different to R₃.

The end-modified poly(β-amino ester) of Formula I may be asymmetric. For example, one of R₁ and R₂ may be an oligopeptide and the other may be R_{y}. Alternatively, R₁ and R₂ may each be a different R_{y}. Alternatively, R₁ and R₂ may each be a different oligopeptide. For example, one of R₁ and R₂ may be an oligopeptide that is positively charged at pH7 and the other of R₁ and R₂ may be an oligopeptide that is negatively charged at pH7. Alternatively, at least one selected from R₁, R₂ and the one or two occurrences of R_{T} may be an oligopeptide and the remaining groups selected from R₁, R₂ and the one or two occurrences of R₁ may be R_{y}. Alternatively, R₁, R₂ and the one or two occurrences of R_{T} may each be a different oligopeptide.

The inventors have found that asymmetric polymers have higher polynucleotide delivery efficiency. For example, polymers according to Formula I wherein one of R₁ and R₂ is CysArgArgArg and the other derived from H₂N(CH₂)₃CH(CH₃)CH₂NH₂ have higher polynucleotide delivery efficiency than both polymers in which both R₁ and R₂ are CysArgArgArg, and polymers wherein both R₁ and R₂ are derived from H₂N(CH₂)₃CH(CH₃)CH₂NH₂.

In nanoparticles according to the present invention, L₃ and L₅ may be independently selected from alkylene, alkenylene, heteroalkylene or heteroalkenylene and polyethylene glycol linkers. Said alkylene, alkenylene, heteroalkylene or heteroalkenylene moieties may be of from 1 to 20 carbon atoms, preferably of from 1 to 12 carbon atoms, more preferably of from 1 to 6 carbon atoms. Said polyethylene glycol linkers may be of 3 to 25 atoms in length, preferably of 3 to 18 atoms in length. Suitably, L₃ is an alkylene of from 3 to 6 carbon atoms.

Optionally, one or more carbon atoms in L₃ and/or L₅ may be replaced with —S—S—. The inclusion of at least one disulfide bond in the main polymer chain allows efficient unpacking of therapeutic polynucleotides inside the target cells.

Suitably, L₄ is

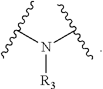

In nanoparticles according to the present invention, each R₃ may be independently selected from the group consisting of hydrogen, —(CH₂)ₚNH₂, —(CH₂)ₚNHMe, —(CH₂)ₚOH, —(CH₂)ₚCH₃, —(CH₂)₂(OCH₂CH₂)_{q}NH₂, —(CH₂)₂(OCH₂CH₂)_{q}OH and —(CH₂)₂(OCH₂CH₂)_{q}CH₃ wherein p is an integer from 1 to 20, for example from 1 to 5, and q is an integer from 1 to 10, for example from 1 to 5. Suitably R₃ is —(CH₂)ₚOH, wherein p is an integer from 1 to 5.

In formula I or II above, n is suitably from 5 to 1000, more suitably from 20 to 500. The molecular weight of the polymer of formula I is suitably from 1,000 to 100,000 g/mol, more suitably 2,000 and 50,000 g/mol more suitably 5,000 and 40,000 g/mol.

Suitably the sugar is a monosaccharide, disaccharide or oligosaccharide. An oligosaccharide may comprise 3 to 10 saccharide units. Suitably the sugar is a disaccharide. Suitably the sugar is sucrose or trehalose.

Suitably the sugar or sugar alcohol is a sugar alcohol. Sugar alcohols have the general formula HOCH₂(CHOH)ₙCH₂OH wherein n is from 0 to 10, suitably from 3 to 4, suitably 4. Suitably, the sugar alcohol is mannitol.

In the invention, weight percent of sugar, sugar alcohol or chitosan is expressed relative to the total weight of the polymer. The weight percent of sugar, sugar alcohol or chitosan has been found to determine the stability of the nanoparticle and the transfection efficiency, and is suitably selected to achieve the appropriate balance of these properties, preferably such that the stability of the nanoparticle is increased or maximised while maintaining high transfection efficiency. Transfection efficiency and the degree of release of the active agent in cells that is observed can be affected by various factors, including high affinity of the nanoparticle for the active agent.

High transfection efficiency can be considered maintained if it is ≧75% of the transfection efficiency of nanoparticles prepared from the polymer without the sugar, sugar alcohol or chitosan. Alternatively, the weight percent can be selected to increase or maximise transfection efficiency while maintaining high stability. High stability can be considered maintained if the mean particle diameter is ≦800 nm after 4 hours incubation at room temperature in phosphate buffer solution at pH7.4

In nanoparticles according to the first aspect of the present invention, the sugar or sugar alcohol is present at 1 to 35 weight percent. Suitably the sugar or sugar alcohol is present at 1 to 18 weight percent, suitably 2 to 15 weight percent, suitably 3 to 12 weight percent, suitably 5 to 10 weight percent. Alternatively, in nanoparticles according to the first aspect of the present invention, the sugar or sugar alcohol is present at 25 to 35 weight percent, suitably 28 to 32 weight percent. Suitably, in nanoparticles according to the first aspect of the invention, the sugar or sugar alcohol is mannitol.

In nanoparticles according to the second aspect of the present invention, the sugar or sugar alcohol is present at 1 to 35 weight percent. Suitably, in nanoparticles according to the second aspect of the present invention, in particular when the sugar or sugar alcohol is mannitol, the sugar or sugar alcohol is present at 1 to 18 weight percent, suitably 3 to 16 weight percent, suitably 8 to 13 weight percent, suitably 9 to 11 weight percent. Alternatively, in nanoparticles according to the second aspect of the present invention, in particular when the sugar or sugar alcohol is sucrose, the sugar or sugar alcohol is present at 25 to 35 weight percent, suitably 28 to 32 weight percent.

Chitosan is a polysaccharide composed of randomly distributed β-(1-4)-linked D-glucosamine (deacetylated unit) and N-acetyl-D-glucosamine (acetylated unit) and is produced commercially from deaceylation of chitin, which is a polysaccharide composed of N-acetyl-D-glucosamine (acetylated unit). In nanoparticles according to the third aspect of the present invention, chitosan is present at 0.15 to 3.0 weight percent, suitably 0.15 to 1.5 weight percent, suitably 0.15 to 0.80 weight percent, suitably 0.25 to 0.75 weight percent, most suitably at 0.50 to 0.70 weight percent. Alternatively, chitosan may be present at 0.30 to 0.40 weight percent. In nanoparticles according to the third aspect of the present invention, the chitosan may have a molecular weight of from 10 to 1000 kg/mol, suitably from 10 to 50 kg/mol, suitably from 15 to 25 kg/mol. Alternatively, the chitosan may have a molecular weight of from 38 to 1000 kg/mol, suitably from 40 to 150 kg/mol, suitably from 40 to 120 kg/mol, suitably from 40 to 100 kg/mol, more suitably from 50 to 80 kg/mol, more suitably from 55 to 65 kg/mol. Alternatively, the chitosan may have a molecular weight of from 60 to 120 kg/mol.

The level of deacetylation of the chitosan may be from 50 to 95%, suitably 50 to 80%, more suitably 55 to 65%. The level of deacetylation is defined as the number of β-(1-4)-linked D-glucosamine residues as a percentage of the total number of residues in the chitosan. Chitosan (or chitin) can be deacetylated by methods known in the art, for example, by reaction with an aqueous sodium hydroxide solution followed by filtering and washing with distilled water until a neutral pH is obtained, and drying (Yuan et al. Materials 4, 1399-1416 (2011)). The degree of deacetylation can be determined using, for example, HPLC, acid base titration or UV-vis spectrophotometry. In the case of UV-vis spectrophotometry, the degree of deacetylation can be determined as follows (Yuan et al. Materials 4, 1399-1416 (2011)): N-acetylglucosamine is dissolved in 0.001 mol/L HCl to prepare 0.1 mg/ml standard solution. A series of 0.01, 0.02, 0.03, 0.04 and 0.05 mg/ml standard solutions is prepared from the 0.1 mg/ml standard solution and the absorbance of each solution at 199 nm is determined on a UV-vis spectrophotometer (Agilent 8453 instrument) using 0.001 mol/l HCl as reference. A standard curve of concentration vs. absorbance is generated. Chitosan (10-20 mg) is dissolved in 10 ml 0.01 mol/l HCl in a 100 ml volumetric flask. After the chitosan is dissolved completely, the solution is diluted to 100 ml using de-ionized water. According to the standard curve, the concentration of acetyl can be determined by measuring the solution absorbance at 199 nm. The degree of deacetylation can be calculated according to the equation:

DDA=100%−C₁/C

where C₁ is the acetyl concentration of sample and C is concentration of sample.

Certain compounds described herein may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2 or 99:1 isomer ratios are all contemplated by the present invention. Those of ordinary skill in the art will readily appreciate that analogous ratios are contemplated for more complex isomer mixtures.

### Chemical Groups

The term “halogen” (or “halo”) includes fluorine, chlorine, bromine and iodine.

The term “alkyl” includes monovalent, straight or branched, saturated, acyclic hydrocarbyl groups. In some embodiments alkyl is C₁₋₁₀alkyl, in another embodiment C₁₋₆alkyl, in another embodiment C₁₋₄alkyl, such as methyl, ethyl, n-propyl, i-propyl or t-butyl groups. Alkyl may be substituted.

The term “cycloalkyl” includes monovalent, saturated, cyclic hydrocarbyl groups. In some embodiments cycloalkyl is C₃₋₁₀ cycloalkyl, in another embodiment C₃₋₆cycloalkyl such as cyclopentyl and cyclohexyl. Cycloalkyl may be substituted.

The term “alkoxy” means alkyl-O—.

The term “alkylamino” means alkyl-NH—.

The term “alkylthio” means alkyl-S(O)ₜ—, wherein t is defined below.

The term “alkenyl” includes monovalent, straight or branched, unsaturated, acyclic hydrocarbyl groups having at least one carbon-carbon double bond and, in some embodiments, no carbon-carbon triple bonds. In some embodiments alkenyl is C₂₋₁₀alkenyl, in another embodiment C₂₋₆ alkenyl, in another embodiment C₂₋₄alkenyl. Alkenyl may be substituted.

The term “cycloalkenyl” includes monovalent, partially unsaturated, cyclic hydrocarbyl groups having at least one carbon-carbon double bond and, in some embodiments, no carbon-carbon triple bonds. In some embodiments cycloalkenyl is C₃₋₁₀ cycloalkenyl, in another embodiment C₅₋₁₀cycloalkenyl, e.g. cyclohexenyl or benzocyclohexyl. Cycloalkenyl may be substituted.

The term “alkynyl” includes monovalent, straight or branched, unsaturated, acyclic hydrocarbyl groups having at least one carbon-carbon triple bond and, in some embodiments, no carbon-carbon double bonds. In some embodiments, alkynyl is C₂₋₁₀ alkynyl, in another embodiment C₂₋₆ alkynyl, in another embodiment C₂₋₄ alkynyl. Alkynyl may be substituted.

The term “alkylene” includes divalent, straight or branched, saturated, acyclic hydrocarbyl groups. In some embodiments alkylene is C₁₋₁₀alkylene, in another embodiment C₁₋₆ alkylene, in another embodiment C₁₋₄alkylene, such as methylene, ethylene, n-propylene, i-propylene or t-butylene groups. Alkylene may be substituted.

The term “alkenylene” includes divalent, straight or branched, unsaturated, acyclic hydrocarbyl groups having at least one carbon-carbon double bond and, in some embodiments, no carbon-carbon triple bonds. In some embodiments alkenylene is C₂₋₁₀alkenylene, in another embodiment C₂₋₆ alkenylene, in another embodiment C₂₋₄ alkenylene. Alkenyene may be substituted.

The term “heteroalkyl” includes alkyl groups, for example, C₁₋₆₅ alkyl groups, C₁₋₁₇alkyl groups or C₁₋₁₀alkyl groups, in which up to twenty carbon atoms, in an embodiment up to ten carbon atoms, in some embodiments up to two carbon atoms, in another embodiment one carbon atom, are each replaced independently by O, S(O)ₜ or N, provided at least one of the alkyl carbon atoms remains. The heteroalkyl group may be C-linked or hetero-linked, i.e. it may be linked to the remainder of the molecule through a carbon atom or through O, S(O)ₜ or N, wherein t is defined below. Heteroalkyl may be substituted.

The term “heterocycloalkyl” includes cycloalkyl groups in which up to ten carbon atoms, in some embodiments up to two carbon atoms, in another embodiment one carbon atom, are each replaced independently by O, S(O)ₜ or N, provided at least one of the cycloalkyl carbon atoms remains.

Examples of heterocycloalkyl groups include oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl and 1,4-diazepanyl. The heterocycloalkyl group may be C-linked or N-linked, i.e. it may be linked to the remainder of the molecule through a carbon atom or through a nitrogen atom. Heterocycloalkyl may be substituted.

The term “heteroalkenyl” includes alkenyl groups, for example, C₁₋₆₅ alkenyl groups, C₁₋₁₇alkenyl groups or C₁₋₁₀alkenyl groups, in which up to twenty carbon atoms, in an embodiment up to ten carbon atoms, in some embodiments up to two carbon atoms, in another embodiment one carbon atom, are each replaced independently by O, S(O)ₜ or N, provided at least one of the alkenyl carbon atoms remains. The heteroalkenyl group may be C-linked or hetero-linked, i.e. it may be linked to the remainder of the molecule through a carbon atom or through O, S(O)ₜ or N. Heteralkenyl may be substituted.

The term “heterocycloalkenyl” includes cycloalkenyl groups in which up to three carbon atoms, in some embodiments up to two carbon atoms, in another embodiment one carbon atom, are each replaced independently by O, S(O)ₜ or N, provided at least one of the cycloalkenyl carbon atoms remains. Examples of heterocycloalkenyl groups include 3,4-dihydro-2H-pyranyl, 5-6-dihydro-2H-pyranyl, 2H-pyranyl, 1,2,3,4-tetrahydropyridinyl and 1,2,5,6-tetrahydropyridinyl. The heterocycloalkenyl group may be C-linked or N-linked, i.e. it may be linked to the remainder of the molecule through a carbon atom or through a nitrogen atom. Heterocycloalkenyl may be substituted.

The term “heteroalkynyl” includes alkynyl groups, for example, C₁₋₆₅ alkynyl groups, C₁₋₁₇alkynyl groups or C₁₋₁₀alkynyl groups, in which up to twenty carbon atoms, in an embodiment in which up to ten carbon atoms, in some embodiments up to two carbon atoms, in another embodiment one carbon atom, are each replaced independently by O, S(O)ₜ or N, provided at least one of the alkynyl carbon atoms remains. The heteroalkynyl group may be C-linked or hetero-linked, i.e. it may be linked to the remainder of the molecule through a carbon atom or through O, S(O)ₜ or N. Heteroalkynyl may be substituted.

The term “heteroalkylene” includes alkylene groups, for example, C₁₋₆₅ alkylene groups, C₁₋₁₇ alkylene groups or C₁₋₁₀alkylene groups, in which up to twenty carbon atoms, in an embodiment in which up to ten carbon atoms, in some embodiments up to two carbon atoms, in another embodiment one carbon atom, are each replaced independently by O, S(O)ₜ or N, provided at least one of the alkylene carbon atoms remains. Heteroalkynylene may be substituted.

The term “heteroalkenylene” includes alkenylene groups, for example, C₁₋₆₅alkenylene groups, C₁₋₁₇alkenylene groups or C₁₋₁₀alkenylene groups, in which up to twenty carbon atoms, in an embodiment in which up to ten carbon atoms, in some embodiments up to two carbon atoms, in another embodiment one carbon atom, are each replaced independently by O, S(O)ₜ or N, provided at least one of the alkenylene carbon atoms remains. Heteroalkenylene may be substituted.

The term “aryl” includes monovalent, aromatic, cyclic hydrocarbyl groups, such as phenyl or naphthyl (e.g. 1-naphthyl or 2-naphthyl). In general, the aryl groups may be monocyclic or polycyclic fused ring aromatic groups. Preferred aryl are C₆-C₁₄aryl. Aryl may be substituted.

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term “arylalkyl” means alkyl substituted with an aryl group, e.g. benzyl.

The term “heteroaryl” includes aryl groups in which one or more carbon atoms are each replaced by heteroatoms independently selected from O, S, N and NR^{N}, where R^{N} is defined below (and in some embodiments is H or alkyl (e.g. C₁₋₆alkyl)). Heteroaryl may be substituted.

In general, the heteroaryl groups may be monocyclic or polycyclic (e.g. bicyclic) fused ring heteroaromatic groups. Typically, heteroaryl groups contain 5-14 ring members (preferably 5-10 members) wherein 1, 2, 3 or 4 ring members are independently selected from O, S, N and NR^{N}. In some embodiments, a heteroaryl group may be 5, 6, 9 or 10 membered, e.g. 5-membered monocyclic, 6-membered monocyclic, 9-membered fused-ring bicyclic or 10-membered fused-ring bicyclic.

Monocyclic heteroaromatic groups include heteroaromatic groups containing 5-6 ring members wherein 1, 2, 3 or 4 ring members are independently selected from O, S, N or NR^{N}.

In some embodiments, 5-membered monocyclic heteroaryl groups contain 1 ring member which is an —NR^{N}— group, an —O— atom or an —S— atom and, optionally, 1-3 ring members (e.g. 1 or 2 ring members) which are ═N— atoms (where the remainder of the 5 ring members are carbon atoms).

Examples of 5-membered monocyclic heteroaryl groups are pyrrolyl, furanyl, thiophenyl, pyrazolyl, imidazolyl, isoxazolyl, oxazolyl, isothiazolyl, thiazolyl, 1,2,3 triazolyl, 1,2,4 triazolyl, 1,2,3 oxadiazolyl, 1,2,4 oxadiazolyl, 1,2,5 oxadiazolyl, 1,3,4 oxadiazolyl, 1,3,4 thiadiazolyl, pyridyl, pyrimidinyl, pyridazinyl, pyrazinyl, 1,3,5 triazinyl, 1,2,4 triazinyl, 1,2,3 triazinyl and tetrazolyl.

Examples of 6-membered monocyclic heteroaryl groups are pyridinyl, pyridazinyl, pyrimidinyl and pyrazinyl.

In some embodiments, 6-membered monocyclic heteroaryl groups contain 1 or 2 ring members which are ═N— atoms (where the remainder of the 6 ring members are carbon atoms).

Bicyclic heteroaromatic groups include fused-ring heteroaromatic groups containing 9-14 ring members wherein 1, 2, 3, 4 or more ring members are independently selected from O, S, N or NR^{N}.

In some embodiments, 9-membered bicyclic heteroaryl groups contain 1 ring member which is an —NR^{N}— group, an —O— atom or an —S— atom and, optionally, 1-3 ring members (e.g. 1 or 2 ring members) which are ═N— atoms (where the remainder of the 9 ring members are carbon atoms).

Examples of 9-membered fused-ring bicyclic heteroaryl groups are benzofuranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolininyl, imidazo[1,2-a]pyridinyl, imidazo[1,5-a]pyridinyl, pyrazolo[1,2-a]pyridinyl, pyrrolo[1,2-b]pyridazinyl and imidazo[1,2-c]pyrimidinyl.

In some embodiments, 10-membered bicyclic heteroaryl groups contain 1-3 ring members which are ═N— atoms (where the remainder of the 10 ring members are carbon atoms).

Examples of 10-membered fused-ring bicyclic heteroaryl groups are quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl and pyrimido[4,5-d]pyrimidinyl.

The term “heteroarylalkyl” means alkyl substituted with a heteroaryl group.

Examples of acyl groups include alkyl-C(═O)—, cycloalkyl-C(═O)—, alkenyl-C(═O)—, cycloalkenyl-C(═O)—, heteroalkyl-C(═O)—, heterocycloalkyl-C(═O)—, aryl-C(═O)— or heteroaryl-C(═O)—, in particular, alkyl-C(═O)— and aryl-C(═O)—.

Unless indicated explicitly otherwise, where combinations of groups are referred to herein as one moiety, e.g. arylalkyl, the last mentioned group contains the atom by which the moiety is attached to the rest of the molecule.

Where reference is made to a carbon atom of an alkyl group or other group being replaced by O, S(O)ₜ or N, what is intended is that:

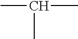

is replaced by

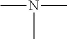

CH═ is replaced by —N═;
≡C—H is replaced by ≡N; or
—CH₂— is replaced by —O—, —S(O)ₜ— or —NR^{N}—.

By way of clarification, in relation to the above mentioned heteroatom containing groups (such as heteroalkyl etc.), where a numerical of carbon atoms is given, for instance C₃₋₆ heteroalkyl, what is intended is a group based on C₃₋₆ alkyl in which one of more of the 3-6 chain carbon atoms is replaced by O, S(O)ₜ or N. Accordingly, a C₃₋₆ heteroalkyl group, for example, will contain less than 3-6 chain carbon atoms.

Where mentioned above, R^{N} is H, alkyl, cycloalkyl, aryl, heteroaryl, —C(O)-alkyl, —C(O)-aryl, —C(O)-heteroaryl, —S(O)ₜ-alkyl, —S(O)ₜ-aryl or —S(O)ₜ-heteroaryl. R^{N} may, in particular, be H, alkyl (e.g. C₁₋₆alkyl) or cycloalkyl (e.g. C₃₋₆ cycloalkyl).

Where mentioned above, t is independently 0, 1 or 2, for example 2. Typically, t is 0.

Where a group has at least 2 positions which may be substituted, the group may be substituted by both ends of an alkylene or heteroalkylene chain to form a cyclic moiety.

It will be appreciated that the compounds described herein may be substituted with any number of substituents or functional moieties. In particular, groups present in the compounds described herein (e.g. alkyl, cycloalkyl, alkoxy, alkenyl, cycloalkenyl, alkynyl, alkylene, alkenylene, heteroalkyl, heterocycloalkyl, heteroalkenyl, heterocycloalkenyl, heteroalkynyl, heteroalkylene, heteroalkenylene, aryl, arylalkyl, arylheteroalkyl, heteroaryl, heteroarylalkyl or heteroarylheteroalkyl groups etc.) may be substituted or unsubstituted, in some embodiments unsubstituted.

The terms substituted, whether preceded by the term “optionally” or not, and substituent, as used herein, refer to the ability, as appreciated by one skilled in this art, to change one functional group for another functional group provided that the valency of all atoms is maintained. Typically, substitution involves the notional replacement of a hydrogen atom with a substituent group, or two hydrogen atoms in the case of substitution by ═O. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions). In some embodiments, substituents are not further substituted.

Where substituted, there will generally be 1 to 3 substituents, in some embodiments 1 or 2 substituents, in some embodiments 1 substituent.

The optional substituent(s) is/are independently halogen, trihalomethyl, trihaloethyl, —OH, —NH₂, —NO₂, —CN, —N⁺(C₁₋₆alkyl)₂O⁻, —CO₂H, —CO₂C₁₋₆ alkyl, —SO₃H, —SOC₁₋₆alkyl, —SO₂C₁₋₆ alkyl, —SO₃C₁₋₆alkyl, —OC(═O)OC₁₋₆alkyl, —C(═O)H, —C(═O)C₁₋₆alkyl, —OC(═O)C₁₋₆ alkyl, ═O, —NH(C₁₋₆ alkyl), —N(C₁₋₆ alkyl)₂, —C(═O)NH₂, —C(═O)N(C₁₋₆alkyl)₂, —N(C₁₋₆ alkyl)C(═O)O(C₁₋₆alkyl), —N(C₁₋₆ alkyl)C(═O)N(C₁₋₆alkyl)₂, —OC(═O)N(C₁₋₆alkyl)₂, —N(C₁₋₆ alkyl)C(═O)C₁₋₆ alkyl, —C(═S)N(C₁₋₆ alkyl)₂, —N(C₁₋₆ alkyl)C(═S)C₁₋₆alkyl, —SO₂N(C₁₋₆alkyl)₂, —N(C₁₋₆ alkyl)SO₂C₁₋₆ alkyl, —N(C₁₋₆alkyl)C(═S)N(C₁₋₆ alkyl)₂, —N(C₁₋₆ alkyl)SO₂N(C₁₋₆alkyl)₂, —C₁₋₆ heteroalkyl, —C₃₋₆ cycloalkyl, —C₃₋₆heterocycloalkyl, —C₂₋₆ alkenyl, —C₂₋₆heteroalkenyl, —C₃₋₆ cycloalkenyl, —C₃₋₆ heterocycloalkenyl, —C₂₋₆ alkynyl, —C₂₋₆ heteroalkynyl, —Z^{u}—C₁₋₆alkyl, —Z^{u}— C₃₋₆cycloalkyl, —Z^{u}—C₂₋₆alkenyl, —Z^{u}—C₃₋₆cycloalkenyl or —Z^{u}—C₂₋₆alkynyl, wherein Z^{u} is independently O, S, NH or N(C₁₋₆alkyl).

In another embodiment, the optional substituent(s) is/are independently halogen, trihalomethyl, trihaloethyl, —NO₂, —CN, —N⁺(C₁₋₆alkyl)₂O⁻, —CO₂H, —SO₃H, —SOC₁₋₆alkyl, —SO₂C₁₋₆alkyl, —C(═O)H, —C(═O)C₁₋₆alkyl, ═O, —N(C₁₋₆alkyl)₂, —C(═O)NH₂, —C₁₋₆ alkyl, —C₃₋₆ cycloalkyl, —C₃₋₆ heterocycloalkyl, —Z^{u}C₁₋₆alkyl or —Z^{u}—C₃₋₆cycloalkyl, wherein Z^{u} is defined above.

In another embodiment, the optional substituent(s) is/are independently halogen, trihalomethyl, —NO₂, —CN, —CO₂H, —C(═O)C₁₋₆alkyl, ═O, —N(C₁₋₆alkyl)₂, —C(═O)NH₂, —C₁₋₆alkyl, —C₃₋₆ cycloalkyl, —C₃₋₆ heterocycloalkyl, —Z^{u}C₁₋₆alkyl or —Z^{u}—C₃₋₆cycloalkyl, wherein Z^{u} is defined above.

In another embodiment, the optional substituent(s) is/are independently halogen, —NO₂, —CN, —CO₂H, ═O, —N(C₁₋₆ alkyl)₂, —C₁₋₆alkyl, —C₃₋₆cycloalkyl or —C₃₋₆heterocycloalkyl.

In another embodiment, the optional substituent(s) is/are independently halogen, —OH, NH₂, NH(C₁₋₆alkyl), —N(C₁₋₆ alkyl)₂, —C₁₋₆ alkyl, —C₃₋₆cycloalkyl or —C₃₋₆ heterocycloalkyl.

As used herein, the terms “chitosan” and “polymer of formula I” etc. include pharmaceutically acceptable derivatives thereof and polymorphs, isomers and isotopically labelled variants thereof.

The term “pharmaceutically acceptable derivative” includes any pharmaceutically acceptable salt, solvate, hydrate or prodrug of a compound described herein. Suitably, the pharmaceutically acceptable derivatives are pharmaceutically acceptable salts, solvates or hydrates of a compound described herein.

The term “pharmaceutically acceptable salt” includes a salt prepared from pharmaceutically acceptable non-toxic acids or bases including inorganic or organic acids and bases.

Compounds described herein which contain basic, e.g. amino, groups are capable of forming pharmaceutically acceptable salts with acids. Pharmaceutically acceptable acid addition salts of the compounds described herein include, but are not limited to, those of inorganic acids such as hydrohalic acids (e.g. hydrochloric, hydrobromic and hydroiodic acid), sulfuric acid, nitric acid and phosphoric acids. Pharmaceutically acceptable acid addition salts of the compounds described herein include, but are not limited to, those of organic acids such as aliphatic, aromatic, carboxylic and sulfonic classes of organic acids, examples of which include: aliphatic monocarboxylic acids such as formic acid, acetic acid, propionic acid or butyric acid; aliphatic hydroxy acids such as lactic acid, citric acid, tartaric acid or malic acid; dicarboxylic acids such as maleic acid or succinic acid; aromatic carboxylic acids such as benzoic acid, p-chlorobenzoic acid, phenylacetic acid, diphenylacetic acid or triphenylacetic acid; aromatic hydroxyl acids such as o-hydroxybenzoic acid, p-hydroxybenzoic acid, 1-hydroxynaphthalene-2-carboxylic acid or 3-hydroxynaphthalene-2-carboxylic acid; and sulfonic acids such as methanesulfonic acid, ethanesulfonic acid or benzenesulfonic acid. Other pharmaceutically acceptable acid addition salts of the compounds described herein include, but are not limited to, those of glycolic acid, glucuronic acid, furoic acid, glutamic acid, anthranilic acid, salicylic acid, mandelic acid, embonic (pamoic) acid, pantothenic acid, stearic acid, sulfanilic acid, algenic acid and galacturonic acid. Wherein the compound described herein comprises a plurality of basic groups, multiple centres may be protonated to provide multiple salts, e.g. di- or tri-salts of compounds described herein. For example, a hydrohalic acid salt of a compound described herein as described herein may be a monohydrohalide, dihydrohalide or trihydrohalide, etc. The salts may include, but are not limited to, those resulting from addition of any of the acids disclosed above. In some embodiments of the compound described herein, two basic groups form acid addition salts. Suitably, the two addition salt counterions are the same species, e.g. dihydrochloride, dihydrosulphide etc. Typically, the pharmaceutically acceptable salt is a hydrochloride salt, such as a dihydrochloride salt.

Compounds described herein which contain acidic, e.g. carboxyl, groups are capable of forming pharmaceutically acceptable salts with bases. Pharmaceutically acceptable basic salts of the compounds described herein include, but are not limited to, metal salts such as alkali metal or alkaline earth metal salts (e.g. sodium, potassium, magnesium or calcium salts) and zinc or aluminium salts. Pharmaceutically acceptable basic salts of the compounds described herein include, but are not limited to, salts formed with ammonia or pharmaceutically acceptable organic amines or heterocyclic bases such as ethanolamines (e.g. diethanolamine), benzylamines, N-methyl-glucamine, amino acids (e.g. lysine) or pyridine.

Hemisalts of acids and bases may also be formed, e.g. hemisulphate salts.

Pharmaceutically acceptable salts of compounds described herein may be prepared by methods well-known in the art.

For a review of pharmaceutically acceptable salts, see Stahl and Wermuth, Handbook of Pharmaceutical Salts: Properties, Selection and Use (Wiley-VCH, Weinheim, Germany, 2002).

The compounds described herein may exist in both unsolvated and solvated forms. The term “solvate” includes molecular complexes comprising a compound described herein and one or more pharmaceutically acceptable solvent molecules such as water or C₁₋₆ alcohols, e.g. ethanol. The term “hydrate” means a “solvate” where the solvent is water.

The compounds described herein may exist in solid states from amorphous through to crystalline forms. All such solid forms are included within the invention.

Compounds described herein may exist in one or more geometrical, optical, enantiomeric, diastereomeric and tautomeric forms, including but not limited to cis- and trans-forms, E- and Z-forms, R-, S- and meso-forms, keto- and enol-forms. All such isomeric forms are included within the invention. The isomeric forms may be in isomerically pure or enriched form, as well as in mixtures of isomers (e.g. racemic or diastereomeric mixtures).

The invention includes pharmaceutically acceptable isotopically labelled compounds described herein wherein one or more atoms are replaced by atoms having the same atomic number, but an atomic mass or mass number different from the atomic mass or mass number usually found in nature.

Examples of isotopes suitable for inclusion in the described herein include isotopes of hydrogen, such as ²H and ³H, carbon, such as ¹¹C, ¹³C and ¹⁴C, chlorine, such as ³⁶Cl, fluorine, such as ¹⁵F, iodine, such as ¹²³I and ¹²⁵I, nitrogen, such as ¹³N and ¹⁵N, oxygen, such as ¹⁵O, ¹⁷O and ¹⁵O, phosphorus, such as ³²P, and sulphur, such as ³⁵S. Certain isotopically-labelled polymers, for example, those incorporating a radioactive isotope, are useful in drug and/or substrate tissue distribution studies. The radioactive isotopes ³H and ¹⁴C are particularly useful for this purpose in view of their ease of incorporation and ready means of detection.

Substitution with positron emitting isotopes, such as ¹¹C, ¹⁸F, ¹⁵O and ¹³N, can be useful in positron emission topography (PET) studies for examining substrate receptor occupancy.

Isotopically labelled compounds described herein can generally be prepared by conventional techniques known to those skilled in the art or by processes analogous to those described herein using an appropriate isotopically-labelled reagent in place of the non-labelled reagent previously employed.

The term thiohydroxyl or thiol, as used herein, refers to a group of the formula —SH.

The present invention further provides nanoparticles comprising two or more different end-modified poly(β-amino ester)s, for example, two of more different polymers as defined in formula I. For example, the nanoparticles may comprise end-modified poly(β-amino ester)s wherein the end-modifying groups are each CysArgArgArg and end-modified poly(β-amino ester)s wherein the end-modifying groups are each CysHisHisHis. Alternatively, the nanoparticles may comprise end-modified poly(β-amino ester)s wherein the end-modifying groups are each CysArgArgArg and end-modified poly(β-amino ester)s wherein the end-modifying groups are each CysAspAspAsp.

The present invention further provides a nanoparticle as described herein, further comprising an active agent. The active agent may be a polynucleotide, protein or small molecule. Typically, the active agent is a polynucleotide. The polynucleotide may be selected from the group consisting of DNA, RNA, siRNA and miRNA, preferably from the group consisting of siRNA and miRNA. Alternatively, the polynucleotide is selected from the group consisting of DNA, RNA and siRNA.

Typically, a polynucleotide comprises at least three nucleotides. Preferably, the polynucleotide is 20-30 nucleotides in length, more preferably 20-25 nucleotides in length, for example, 22 nucleotides in length.

The polynucleotide may be derived from natural nucleosides (i.e., adenosine, thymidine, guanosine, cytidine, uridine, inosine, xanthosine, deoxyadenosine, deoxythymidine, deoxyguanosine, deoxyinosine, and deoxycytidine), nucleoside analogues (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), or mixtures thereof. The nucleotides may be derived from chemically modified bases, biologically modified bases (e.g., methylated bases), intercalated bases, unmodified or modified sugars (e.g., 2′-fluororibose, ribose, 2′-deoxyribose, arabinose, and hexose), and/or unmodified or modified phosphate groups (e.g., phosphorothioates and 5′-N-phosphoramidite linkages).

As used herein, the term “small molecule” refers to organic compounds, whether naturally-occurring or artificially created (e.g., via chemical synthesis) that have relatively low molecular weight and that are not proteins, polypeptides, or polynucleotides. Typically, small molecules have a molecular weight of less than about 1500 g/mol.

Further discussion of the nanoparticles of the present invention follows. It will be understood that the discussion also applies to microparticles.

The nanoparticles may comprise a polynucleotide and an end-modified poly(β-amino ester) wherein at least one end modification is an oligopeptide having a net positive charge at pH 7. The positively charged oligopeptides interact with negatively charged polynucleotide during the process of nanoparticle formation and facilitate encapsulation of the polynucleotide in the nanoparticles.

The nanoparticles may comprise an active agent that has a net positive charge at pH7 and an end-modified poly(β-amino ester) wherein at least one end modification is an oligopeptide has a net negative charge at pH 7. The negatively charged oligopeptides interact with positively charged active agent during the process of nanoparticle formation and facilitate encapsulation of the active agent in the nanoparticles.

Nanoparticles according to the invention may optionally comprise a mixture of different polymers of formula I. For example, nanoparticles may comprise

(a) a polymer according to formula I wherein the or each oligopeptide has a net positive charge at pH 7; and

(b) a polymer according to formula I wherein the or each oligopeptide has a net negative charge at pH 7.

Thus, the invention provides nanoparticles with net surface charge that may be varied by modifying the proportions of polymers (a) and (b) above. The ratio of (a) to (b) may be 1:99, 5:95, 10:90, 25:75, 50:50, 75:25, 90:10, 95:5, or 99:1 by weight.

Such nanoparticles are suitable for both drug and polynucleotide encapsulation and show improved pharmacological properties.

The inclusion of a population of poly(β-amino ester)s end modified with oligopeptides that have a net negative charge at pH 7 facilitates encapsulation by nanoprecipitation of shorter DNA and RNA sequences. Shorter DNA and RNA sequences show lower encapsulation efficiency and/or lower absolute loading than longer sequences when used in a nanoprecipitation step with PBAEs known in the art. The inventors have found that the addition of other polyanionic species, such as the negatively charged polymers described here, helps in the assembly during the nanoprecipitation process of the resulting nanoparticles containing polymer and polynucleotide.

This is especially useful for the encapsulation of short polynucleotides, such as siRNA and miRNA sequences, which have a length of approximately 20 to 30 base pairs and are unstable during circulation in the body. The incorporation of short polynucleotides such as siRNA and miRNA into nanoparticles has previously presented difficulties owing to their lower charge.

The use of poly(β-amino ester)s end modified with an oligopeptide having a net positive charge at pH 7 in combination with poly(β-amino ester)s end modified with an oligopeptide having a net negative charge at pH 7 allows the loading of short polynucleotides such as siRNA or miRNA into nanoparticles with high encapsulation efficiency and high loading. Further, the use of the two types of polymers described above prevents degradation of the short polynucleotides and allows more efficient transfection. It is thought that the positively charged oligonucleotides “wrap” around the negatively charged polynucleotides, and the negatively charged oligonucleotides “wrap” around the positively charged oligonucleotides to neutralize the excess charge (referred to by the inventors as the “mantle effect”).

Further, the inclusion of, poly(β-amino ester)s end modified with oligopeptides that have a net negative charge at pH 7 facilitates delivery of the nanoparticles through complex body barriers, such as intestinal and pulmonary mucosa, as the net surface charge changes may vary during the interaction with those barriers.

Nanoparticles of the present invention may be formed with high active agent content and high active agent encapsulation efficiency.

Herein, the active agent encapsulation efficiency refers to the active agent incorporated into the nanoparticles as a weight percentage of the total active agent used in the method of preparation of the active agent-containing nanoparticles. It is typically up to and including 95%, more typically from 70% to 95%.

Herein, active agent content refers to the weight percentage of the active agent in the active agent-loaded nanoparticles. Active agent content is preferably at least 2 wt %, more preferably at least 5 wt %, more preferably at least 10 wt % and typically in the range of from 2 wt % to 20 wt %, more preferably from 5 wt % to 20 wt %, more preferably from 10 wt % to 20 wt %.

The active agent(s) may be present within the nanoparticles or on the surfaces of the nanoparticles. Typically the active agent(s) are present within the nanoparticles. The interaction between the active agent(s) and the nanoparticle is typically non-covalent, for example, hydrogen bonding, electrostatic interaction or physical encapsulation. Typically the interaction is electrostatic

The nanoparticles are biocompatible and sufficiently resistant to their environment of use that a sufficient amount of the nanoparticles remain substantially intact after entry into the mammalian body so as to be able to reach the desired target and achieve the desired physiological effect. The polymers described herein are biocompatible and preferably biodegradable.

Herein, the term ‘biocompatible’ describes a substance which may be inserted or injected into a living subject without causing an adverse response. For example, it does not cause inflammation or acute rejection by the immune system that cannot be adequately controlled. It will be recognized that “biocompatible” is a relative term, and some degree of immune response is to be expected even for substances that are highly compatible with living tissue. An in vitro test to assess the biocompatibility of a substance is to expose it to cells; biocompatible substances will typically not result in significant cell death (for example, >20%) at moderate concentrations (for example, 29 μg/10⁴ cells).

Herein, the term ‘biodegradable’ describes a polymer which degrades in a physiological environment to form monomers and/or other non-polymeric moieties that can be reused by cells or disposed of without significant toxic effect. Degradation may be biological, for example, by enzymatic activity or cellular machinery, or may be chemical, typically a chemical process that takes place under physiological conditions. Degradation of a polymer may occur at varying rates, with a half-life in the order of days, weeks, months, or years, depending on the polymer or copolymer used. The components preferably do not induce inflammation or other adverse effects in vivo. Suitably, the chemical reactions relied upon to break down the biodegradable compounds are uncatalysed.

Herein, the term ‘nanoparticle’ refers to a particle with a diameter of from about 1 to about 1000 nm. Herein, the term ‘microparticle’ refers to a particle with a diameter of from greater than about 1 μm to about 100 μm. The nanoparticle or microparticle is suitably a polyplex, that is, a complex of polymer(s) and, optionally, an active agent.

The mean diameter of the nanoparticles of the present invention may be determined by methods known in the art, preferably by dynamic light scattering (DLS). In particular, the invention relates to nanoparticles that are solid particles with a diameter of from about 1 to about 1000 nm when analysed by DLS at a scattering angle of 90° and at a temperature of 25° C., using a sample appropriately diluted with filtered water and a suitable instrument such as the Zetasizer™ instruments from Malvern Instruments (UK) according to the standard test method ISO 22412:2008 (cumulants method A.1.3.2). Where a particle is said to have a diameter of x nm, there will generally be a distribution of particles about this mean, but at least 50% by number (e.g. >60%, >70%, >80%, >90%, or more) of the particles will have a diameter within the range x±20%.

Preferably, the diameter of the nanoparticle is from about 10 to about 1000 nm, more preferably from about 5 to about 500 nm, more preferably from about 50 to about 400 nm, more preferably from about 50 to about 150 nm. Alternatively, the diameter of the nanoparticle is from about 1 to about 100 nm. The nanoparticles suitably exhibit a degree of agglomeration of less than 10%, preferably less than 5%, preferably less than 1%, and preferably the nanoparticles are substantially non-agglomerated, as determined by transmission electron microscopy.

The nanoparticles of the present invention may be produced by methods known in the art, which may be divided into two main categories: (i) formation including a polymerization reaction; and (ii) formation by dispersion of a preformed copolymer.

Formation of nanoparticles including a polymerization reaction can be further classified into emulsion and interfacial polymerization. Emulsion polymerization may be organic or aqueous, depending on the continuous phase.

Formation of nanoparticles by dispersion of a preformed polymer can include the following techniques: emulsification/solvent evaporation, solvent displacement and interfacial deposition, emulsification/solvent diffusion, and precipitation by increasing salt concentration. In these techniques, the polymer is first produced then processed further to form the nanoparticles.

The methods may utilize interfacial condensation, supercritical fluid processing techniques, ionic gelation or coacervation for the production of the nanoparticles.

The solvent displacement method (Fessi et al. Int. J. Pharmaceutics 55, R1-R4(1989)) has been used for the formation of nanoparticles. Bilati et al. (Eur. J. Pharm. Sci. 24, 67-75 (2004)) describes the approaches that have been taken to achieve encapsulation of hydrophilic drugs by this method.

The solvent displacement method does not require high stirring rates, sonication or very high temperatures. For example, it may be carried out at 25° C. and at stirring rates of 50-150 rpm, more preferably about 100 rpm. It is characterized by the absence of an oily-aqueous interface, reducing the likelihood of damage to the active agent(s). The procedure may be carried out without use of surfactants, and without the use of organic solvents that may be toxic and therefore incompatible with pharmaceutical and veterinary applications if residues in excess of acceptable limits remain in the nanoparticles.

The solvent displacement method uses two solvents that are miscible and constitute a diffusing medium and a dispersing medium. Preferably, the polymer and, if present, the active agent(s) are soluble in the diffusing medium (typically referred to as “the solvent”) but neither is soluble in the dispersing medium (typically referred to as “the non-solvent”). The polymer and optionally the active agent(s) are dissolved in the diffusing medium and the resulting solution is added to the dispersing medium. Optionally, the dispersing medium includes a surfactant. As soon as the diffusing medium has diffused into the dispersing medium, nanoprecipitation occurs by a rapid desolvation of the polymer, forming nanoparticles in which the active agent is sited within the copolymer. The diffusing medium is preferably added directly to the dispersing medium, for example via syringe, in order to avoid introduction of an air-liquid interface into the process. Various methods are available for separating the nanoparticles from the dispersing and diffusing media, for example, lyophilization, tangential filtration, centrifuge and ultra-centrifuge, or a combination of these methods. In some cases, for example when the nanoparticles are large, centrifugation is preferred. In some cases, for example in the preparation of large batches, the nanoparticle composition may be concentrated by tangential filtration then lyophilized. Preferably, the dispersing and diffusing media are removed by centrifugation or rotary evaporation. The particles are optionally resuspended in a solvent to remove adhered active agent from the surface of the nanoparticles. This solvent may be removed by a further centrifugation step. The nanoparticles may finally be resuspended in a suitable polar liquid.

Where the nanoparticles of the present invention comprise an active agent, the active agent may be present during the production of the nanoparticles, typically wherein the active agent(s) are present in a liquid medium used for the production of the nanoparticles. Alternatively, or additionally, the active agent(s) may be incorporated by absorption into the nanoparticles after their production.

Suitably, the nanoparticles are formed by mixing, for example, with vortex, a solution comprising the required materials then incubating the solution. For example, nanoparticles may be formed by mixing, for example, with vortex, a solution comprising the PBAE with a solution comprising an active agent such as a nucleic acid, then incubating the solution. Suitably, the solution(s) contains a buffer such as sodium acetate buffer. The final concentration of sodium acetate buffer may be from 2 to 15 mM, suitably form 10 to 13 mM, suitably from 11 to 12 mM. Suitably, the solution is incubated for at least 5 minutes, suitably for 5 to 20 minutes. The solution may be incubated at 10 to 40° C., suitably at 17 to 25° C.

The present invention further provides a composition comprising at least one nanoparticle described herein. Preferably, the nanoparticles constitute from about 1% to about 90% by weight of the composition. More preferably, the nanoparticles constitute about 5% to about 50% by weight of the composition, more preferably, about 10% to about 30%. The composition may be a pharmaceutical composition for mammalian and particularly human use. The composition may further comprise a vehicle. The vehicle may be any pharmaceutically acceptable diluent or excipient, as known in the art. The vehicle is typically pharmacologically inactive. Preferably, the vehicle is a polar liquid. Particularly preferred vehicles include water and physiologically acceptable aqueous solutions containing salts and/or buffers, for example, saline or phosphate-buffered saline. Optionally, the vehicle is a biological fluid. A liquid vehicle may be removed by, for example, lyophilization, evaporation or centrifugation for storage or to provide a powder for pulmonary or nasal administration, a powder for suspension for infusion, or tablets or capsules for oral administration.

The present invention further provides a method of encapsulating an active agent in a matrix of chitosan and a polymer of formula I to form nanoparticles, the method comprising steps of providing an active agent; providing a mixture of chitosan and the polymer; and contacting the active agent and the polymer under suitable conditions to form nanoparticles. In particular, the components may be mixed in solution at concentrations appropriate to obtain the desired ratio, mixed vigorously and then incubated. Suitably, the solution(s) contains a buffer such as sodium acetate buffer. Suitably, the solution is incubated for at least 5 minutes, suitably for 5 to 20 minutes. The solution may be incubated at 10 to 40° C., suitably at 17 to 25° C.

The present invention further provides a method of coating a polymeric nanoparticle with a sugar or sugar alcohol, the method comprising the step of contacting a polymeric nanoparticle with a sugar or sugar alcohol. The sugar or sugar alcohol, the required weight percent of the sugar or sugar alcohol relative to the weight of polymer, and the polymer are suitably those disclosed hereinabove.

A polymer of formula I may be synthesized by a method comprising the steps of reacting a compound of Formula II, wherein L₃ is as defined above, with a primary amine of formula L₄H₂, wherein L₄ is as defined above, to produce a polymer of Formula III as shown below.

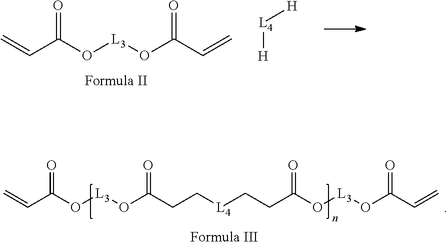

The compound of Formula III is further reacted with compounds of Formula IV to form a compound of Formula V:

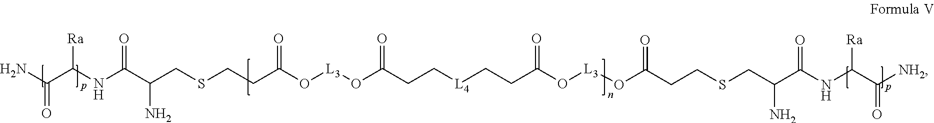

wherein p and Rₐ independently at each occurrence are selected from the lists defined above. In some cases, each occurrence of p is the same and the Rₐ groups are selected such that the sequence of Rₐ groups starting from the sulfur linkage is the same at each end of the compound, that is, p and Rₐ are selected such that the polymer has two-fold symmetry about L₄.

In an alternative to the above step, the compound of Formula III is further reacted with compounds of formula H₂NR_{y}, wherein R_{y} is as defined above, and compounds of Formula IV and the resulting mixture is separated to obtain a compound of Formula VI:

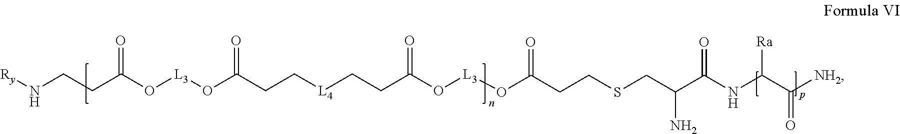

wherein Rₐ is independently selected at each occurrence from the lists defined above and p is as defined above.

It will be recognized that further methods of attaching an oligopeptide to the compound of Formula III would be available to the skilled person, who would be aware of appropriate nucleophiles for reaction at the terminal acrylate groups of Formula III.

The present invention further provides a nanoparticle or a composition described herein for use in medicine. In particular, the nanoparticle or composition may be used in a method of treatment of a disease or disorder caused by overexpression of a protein. For example, the nanoparticle or composition may be used in a method of treatment of cancer, monogenic diseases, vascular disease and infectious diseases, particularly infectious diseases caused by viruses.

The present invention further provides an in vitro method of inhibiting gene expression comprising contacting one or more cells or a tissue with a nanoparticle or a composition described herein.

The invention is further illustrated by the following examples. It will be appreciated that the examples are for illustrative purposes only and are not intended to limit the invention as described above. Modification of detail may be made without departing from the scope of the invention.

### BRIEF DESCRIPTION OF THE FIGURES

FIG. 1 shows the mean diameter and zeta potential of nanoparticles according to the first aspect of the invention formulated with mannitol, trehalose and sucrose at various weight percents.

FIG. 2 shows the mean diameter and zeta potential of nanoparticles according to the third aspect of the invention coated with chitosan at various weight percents.

FIG. 3 shows a gel retardation assay of R/DNA coated with different molecular weights of chitosan at various weight percents.

FIG. 4 shows the mean diameter and zeta potential of nanoparticles according to the second aspect of the invention coated with mannitol, trehalose and sucrose at various weight percents.

FIG. 5 shows the mean size of nanoparticles in the presence of buffers of differing ionic strength and/or prepared by different procedures.

FIG. 6 shows the change in particle size over time for nanoparticles with differing amounts of chitosan.

FIG. 7 shows a second experiment monitoring the change in particle size over time for nanoparticles with differing amounts of chitosan.

FIG. 8 shows the change in particle size over time for nanoparticles with differing amounts of chitosan having different molecular weights.

FIG. 9 shows the change in count rate (in kilo counts per second) over time for nanoparticles with differing amounts of chitosan.

FIG. 10 shows the mean diameter and zeta potential of R/DNA nanoparticles coated with different molecular weights of chitosan at various weight percents.

FIG. 11 the effect on stability of coating nanoparticles with differing amounts of mannitol.

FIG. 12 shows the effect on stability of coating nanoparticles with mannitol, trehalose or sucrose.

FIG. 13 shows the change in particle size over time for nanoparticles coated with specific amounts of mannitol, trehalose and sucrose

FIG. 14 shows the change in particle size over time for three of the nanoparticles described in FIG. 12.

FIG. 15 shows the change in particle size over time for nanoparticles formulated with 20% mannitol or 20% sucrose.

FIG. 16 shows the change in particle size over time for nanoparticles coated with mannitol, formulated with mannitol, or without mannitol.

FIG. 17 shows the change in particle size over time for nanoparticles formulated with 20 weight percent mannitol.

FIG. 18 shows GFP expression in NRK-52e cells as analysed by flow cytometry, 48 hours after transfection with nanoparticles having differing amounts of chitosan.

FIG. 19 shows GFP expression in NRK-52e cells as analysed by fluorescence microscopy, 48 hours after transfection with nanoparticles having differing amounts of chitosan.

FIG. 20 shows GFP expression in NRK-52e cells as analysed by flow cytometry, 48 hours after transfection with nanoparticles having differing mixtures of PBAEs with or without chitosan.

FIG. 21 shows GFP expression in NRK-52e cells as analysed by fluorescence microscopy, 48 hours after transfection with nanoparticles having differing mixtures of PBAEs with or without chitosan.

FIG. 22 shows transfection efficacy of complexes with or without coating agents in COS-7 cells. (A) Fluorescent images of GFP expression in COS-7 cells: (i) RD; (ii) R/CS0.17; (iii) R/CSM0.17. (B) Percentage of GFP positive cells multiplied by the GeoMean fluorescence of the positive population. (C) GFP expression was determined after 48 h by flow cytometry.

FIG. 23 shows GFP expression in COS-7 cells after transfection with MntR20 nanoparticles prepared using different incubation conditions and durations.

FIG. 24 shows GFP expression in COS-7 cells after transfection with nanoparticles according the invention with different weight percents of additives.

FIG. 25 shows viability of cells after transfection with PBAE/DNA complexes modified with sugar or sugar alcohol.

The invention is further illustrated by the following examples. It will be appreciated that the examples are for illustrative purposes only and are not intended to limit the invention as described above. Modification of detail may be made without departing from the scope of the invention.

### EXAMPLES

### Materials and Methods

Reagents and solvents were obtained from Sigma-Aldrich and Panreac and used as received unless otherwise stated. H-Cys-Arg-Arg-Arg-NH2.4HCl (CR3.4HCl) was obtained from GL Biochem Ltd. (Shanghai, China). Chitosan with molecular weight 22 kg/mol and deacetylation degree 85% was purchased from Fluka. Chitosan with molecular weight 20-50 kg/mol and deacetylation degree 85% was purchased from Creative PEGWorks. Chitosan with molecular weight 60 kg/mol and deacetylation degree 60% was purchased from Sigma Aldrich. Chitosan with molecular weight 60-120 kg/mol and deacetylation degree 60% was purchased from Sigma Aldrich. Oligopeptides were obtained from GL Biochem Ltd. (Shanghai, China). Plasmid encoding green fluorescent protein (pmaxGFP, 3486 bp) was obtained from Amaxa (Gaithersburg, Md., USA). Cell lines were obtained from ATCC (Manassas, Va.) and maintained at 37° C. in 5% CO2 atmosphere in complete DMEM, containing 10% fetal bovine serum, 100 units/ml penicillin, 100 μg/ml streptomycin, 0.1 mM MEM Non-Essential Amino Acids (NEAA), 2 mM L-glutamine obtained from Gibco. All reagents were analytical grade and used without further purification.

Unless otherwise noted, the mean diameter, zeta potential and polydispersity of nanoparticles were determined, diluted in PBS to a final concentration of 0.25 mg/ml, by DLS using a Zetasizer nano zs90 (Malvern Instruments) at 25° C. Each experiment was carried out in triplicate and the mean result was reported.

¹H-NMR spectra were acquired at 25° C. on a Varian NMR instrument operating at 400 MHz with samples dissolved in either deuterated dimethyl sulfoxide (d6-DMSO) or deuterated methanol (CD₃OD) and using tetramethylsilane (TMS) as internal reference. 8-10 mg of sample dissolved in 1 ml of solvent was used for ¹H-NMR.

IR spectra were obtained using a Nicolet Magna 560 (Thermo Fisher Scientific, Waltham, Mass.) with a KBr beamsplitter, using methanol as solvent in evaporated film.

Molecular weight was determined by gel permeation chromatogram carried out at 35° C. with a refraction-index detector with 0.05 M tetrahydrofuran (THF) as mobile phase using a GPC KF-603 column with a flow rate of 0.5 ml/min. Chromatograms were calibrated against polystyrene monodisperse standards.

### Example 1: Synthesis of PBAE Polymers

Acrylate-terminated poly(β-amino ester) C32 was obtained by mixing 1,4-butanediol diacrylate (8.81 g, 40 mmol) and 5-amino-1-pentanol (3.44 g, 33 mmol) in Teflon-lined screw cap vials. The mixture was left under stirring at 90° C. for 24 h. Then it was cooled to room temperature to form a transparent yellow viscous solid, C32, and was stored at −20° C. used for each experiment.

Weight-average molecular weight 2100 g/mol.

Number-average molecular weight 1320 g/mol.

¹H NMR of C32 (d6-DMSO): δ (ppm) 1.2-1.4 (m, —NCH₂(CH₂)₃CH₂OH), 1.6 (br, —N(CH₂)₂COOCH₂CH₂— and CH₂CHCOOCH₂CH₂—), 2.3-2.4 (m, —COOCH₂CH₂N— and —NCH₂(CH₂)₄OH), 2.6 (m, —COOCH₂CH₂N—), 3.4 (br, —N(CH₂)₄CH₂OH), 4.0 (br, —N(CH₂)₂COOCH₂CH₂—), 4.1 (m, CH₂CHCOOCH₂CH₂—), 4.3 (br, —N(CH₂)₅OH), 5.9 (m, CH₂CHCOO CH₂CH₂—), 6.1-6.2 (m, CH₂CHCOOCH₂CH₂—), 6.3-6.4 (m, CH₂CHCOO CH₂CH₂—).

### Example 2: Synthesis of PBAEs End Modified with Oligopeptides

In general, oligopeptide-modified PBAEs may be obtained as follows: acrylate-terminated polymer C32 or C32SS and either amine- or thiol-terminated oligopeptide (for example, HS-Cys-Arg-Arg-Arg (CR3), H₂N-Arg-Arg-Arg (R3) or HS-Cys-Glu-Glu-Glu (CE3)—other oligopeptides are indicated by similar abbreviations using the standard one-letter code) were mixed at 1:2.5 molar ratio in DMSO. The mixture was stirred overnight at room temperature and the resulting polymer may be obtained by precipitation in diethyl ether:acetone (3:1). The polymers may then be dissolved at 100 mg/ml in DMSO and stored at −20° C. until further use.

(a) In a typical example, for the R polymer, C32 (150 mg, 0.07 mmol), CR3.4HCl (115 mg, 0.18 mmol) and DMSO (3 ml) were placed in Teflon-lined screw cap vials and stirred at room temperature for 24 h. End-modified polymer, R3C-C32-CR3 (R) was purified by precipitation in diethyl ether/acetone (7:3 v/v) for twice and dried under vacuum. Dried polymers were finally dissolved at 100 mg/ml in DMSO and stored at −20° C. until further use.

The chemical structure of the oligopeptide-modified PBAEs was confirmed in ¹H-NMR spectroscopy by the disappearance of acrylate signals and the presence of signals typically associated with amino acid moieties.

¹H-NMR of R (400 MHz, CD₃OD, TMS) (ppm): δ=4.43-4.34 (br, NH₂—C(═O)—CH—NH—C(═O)—CH—NH—C(═O)—CH—NH—C(═O)—CH—CH₂—), 4.17 (t, CH₂—CH₂—O), 3.57 (t, CH₂—CH₂—OH), 3.22 (br, NH₂—C(═NH)—NH—CH₂—, OH—(CH₂)₄—CH₂—N—), 2.83 (dd, —CH₂—S—CH₂), 2.75 (m, CH₂—CH₂—N—), 2.48 (br, —N—CH₂—CH₂—C(═O)—O), 1.90 (m, NH₂—C(═NH)—NH—(CH₂)₂—CH₂—CH—), 1.73 (br, —O—CH₂—CH₂—CH₂—CH₂—O), 1.69 (m, NH₂—C(═NH)—NH—CH₂—CH₂—CH₂—), 1.56 (br, —CH₂—CH₂—CH₂—CH₂—OH), 1.39 (br, —N—(CH₂)₂—CH₂—(CH₂)₂—OH).

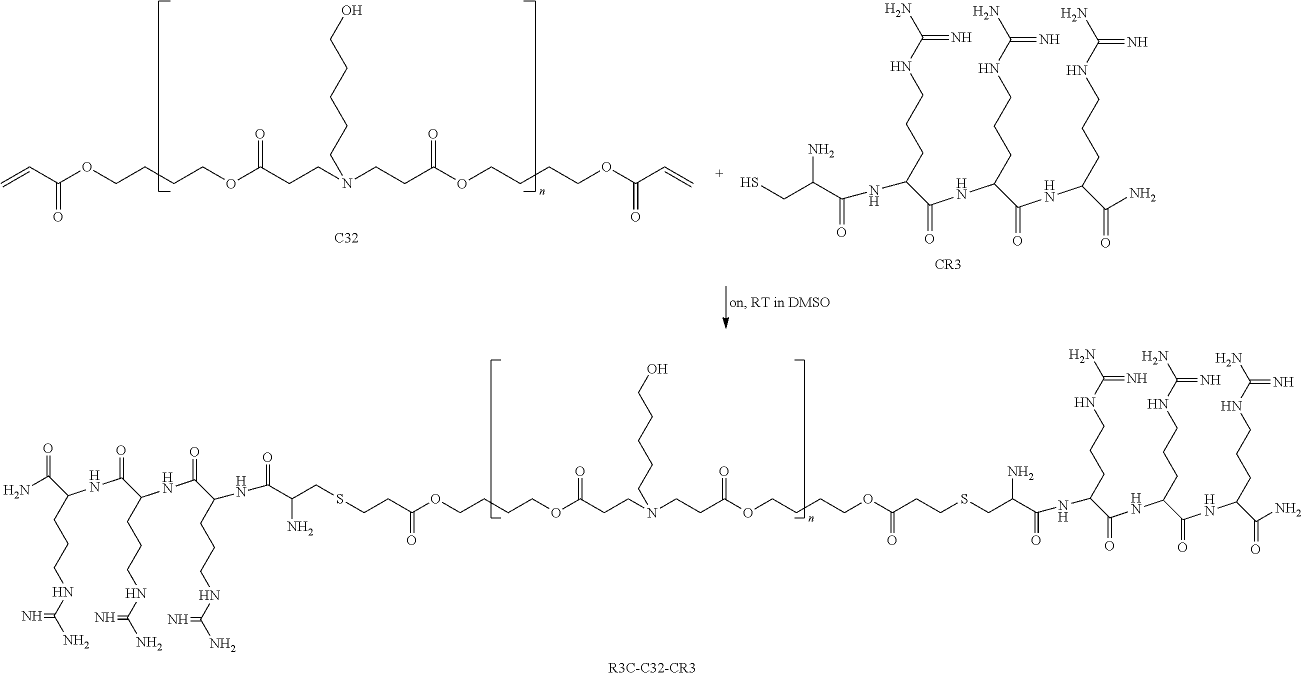

IR (evaporated film): ν=721, 801, 834, 951, 1029, 1133 (C—O), 1201, 1421, 1466, 1542, 1672 (C═O, from peptide amide), 1731 (C═O, from ester), 2858, 2941, 3182, 3343 (N—H, O—H) cm⁻¹

¹H-NMR (400 MHz, CD₃OD, TMS) (ppm): δ=4.41-4.33 (br, NH₂—C(═O)—CH—NH—C(═O)—CH—NH—C(═O)—CH—NH—C(═O)—CH—CH₂—, 4.11 (t, CH₂—CH₂—O), 3.55 (t, CH₂—CH₂—OH), 3.22 (br, NH₂—C(═NH)—NH—CH₂—, OH—(CH₂)₄—CH₂—N—), 3.04 (t, CH₂—CH₂—N—), 2.82 (dd, —CH₂—S—CH₂), 2.48 (br, —N—CH₂—CH₂—C(═O)—O), 1.90 (m, NH₂—C(═NH)—NH—(CH₂)₂—CH₂—CH—), 1.73 (br, —O—CH₂—CH2-CH2-CH₂—O), 1.69 (m, NH₂—C(═NH)—NH—CH₂—CH₂—CH₂—), 1.56 (br, —CH₂—CH₂—CH₂—CH₂—OH), 1.39 (br, —N—(CH₂)₂—CH₂—(CH₂)₂—OH).

(b) in a further example, tri-lysine modified oligopeptides (K3C-C32-CK3) was prepared by mixing a solution of intermediate C32 in DMSO (2 ml) with the corresponding solution of oligopeptide (Cys-Lys-Lys-Lys (CK3) in DMSO (1 ml) in an appropriate molar ratio, 1:2 respectively. The mixture was stirred overnight at room temperature, then precipitated in diethyl ether/acetone (3:1).

IR (evaporated film): ν=721, 799, 834, 1040, 1132, 1179 (C—O), 1201, 1397, 1459, 1541, 1675 (C═O, from peptide amide), 1732 (C═O, from ester), 2861, 2940, 3348 (N—H, O—H) cm⁻¹

¹H-NMR (400 MHz, CD₃OD, TMS) (ppm): δ=4.38-4.29 (br, NH₂—(CH₂)₄—CH—), 4.13 (t, CH₂—CH₂—O—), 3.73 (br, NH₂—CH—CH₂—S—), 3.55 (t, CH₂—CH₂—OH), 2.94 (br, CH₂—CH₂—N—, NH₂—CH₂—(CH₂)₃—CH—), 2.81 (dd, —CH₂—S—CH₂), 2.57 (br, —N—CH₂—CH₂—C(═O)—O), 1.85 (m, NH₂—(CH₂)₃—CH₂—CH—), 1.74 (br, —O—CH₂-CH2-CH2-CH₂—O), 1.68 (m, NH₂—CH₂—CH₂—(CH₂)₂—CH—), 1.54 (br, —CH₂—CH₂—CH₂—CH₂—OH), 1.37 (br, —N—(CH₂)₂—CH₂—(CH₂)₂—OH).

(c) Tri-histidine modified oligopeptides (H3C-C32-CH3) were prepared according to the same protocol as K3C-C32-CK3 and characterized as follows:

IR (evaporated film): ν=720, 799, 832, 1040, 1132, 1201, 1335, 1403, 1467, 1539, 1674 (C═O, from peptide amide), 1731 (C═O, from ester), 2865, 2941, 3336 (N—H, O—H) cm⁻¹

¹H-NMR (400 MHz, CD₃OD, TMS) (ppm): 6=8.0-7.0 (br —N(═CH)—NH—C(═CH)—) 4.61-4.36 (br, —CH2-CH—), 4.16 (t, CH2-CH2-O—), 3.55 (t, CH2-CH2-OH), 3.18 (t, CH2-CH₂—N—, 3.06 (dd, —CH2-CH—), 2.88 (br, OH—(CH₂)₄—CH₂—N—), 2.82 (dd, —CH₂—S—CH₂—), 2.72 (br, —N—CH₂—CH₂—C(═O)—O), 1.75 (br, —O—CH₂-CH2-CH2-CH₂—O), 1.65 (m, NH₂—CH₂—CH₂—(CH₂)₂—CH—), 1.58 (br, —CH₂—CH₂—CH₂—CH₂—OH), 1.40 (br, —N—(CH₂)₂—CH₂—(CH₂)₂—OH).

### Example 3: Synthesis of PBAEs with Asymmetric End Modifications

In general, asymmetric oligopeptide-modified PBAEs were obtained as follows: Acrylate-terminated polymer C32 (or C32SS) and either amine- or thiol-terminated oligopeptide (for example, CR3, R3 or CE3) were mixed at 1:1 molar ratio in DMSO. The mixture was stirred overnight at room temperature. Equimolar amount of a second amine- or thiol-terminated oligopeptide, or of a primary amine, was added and the mixture was stirred overnight at room temperature. The resulting asymmetric PBAE polymers were obtained by precipitation in diethyl ether/acetone (3:1). The following synthetic procedure to obtain asymmetric end-modified B3-C32-CR3 PBAEs is shown as an example: a solution of intermediate C32 (0.15 g, 0.075 mmol) in DMSO (2 ml) was mixed with the corresponding solution of oligopeptide Cys-Arg-Arg-Arg (CR3; 0.055 g, 0.075 mmol) in DMSO (1 ml) and was stirred overnight at room temperature. Subsequently, 2-methyl-1,5-pentanediamine (0.017 g, 0.02 ml, 0.15 mmol) was added in the mixture for 4h at room temperature in DMSO. A mixture of asymmetric end-modified polymer B3-C32-CR3 with B3-C32-B3 and R3C-C32-CR3 was obtained by precipitation overnight in diethyl ether/acetone (3:1). The mixture may be used without further purification or the asymmetric end-modified polymer B3-C32-CR3 may be separated from the mixture by standard methods.

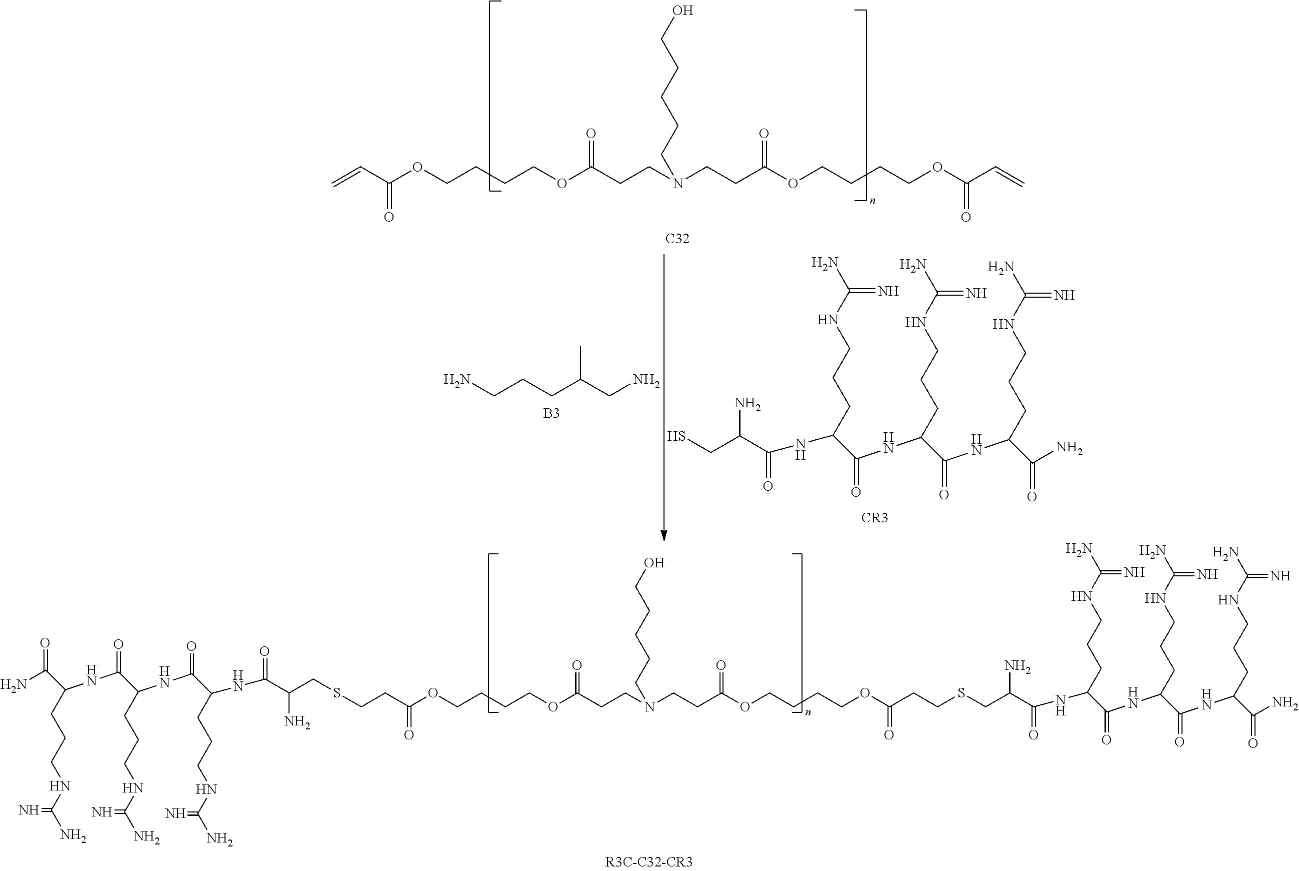

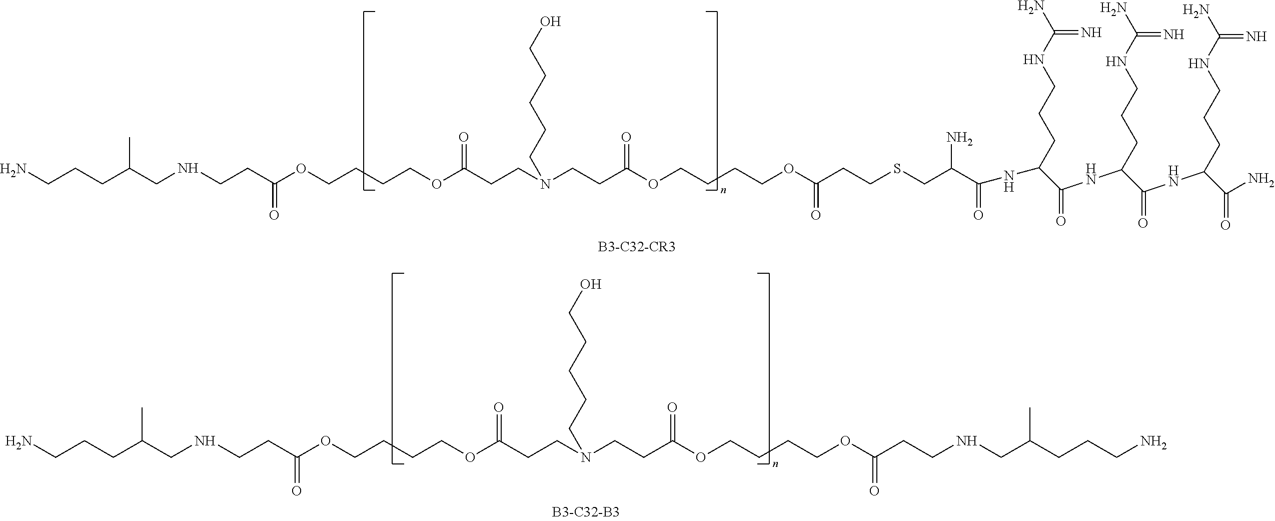

### Example 4: Library of Compounds

A library of different oligopeptide end-modified PBAEs was synthesized by adding primary amines to diacrylates followed by end-modification. According to Formula I, the oligopeptide end-modified PBAEs shown in Table 1 were synthesized.

TABLE 1
Library of oligopeptide end-modified PBAEs

| Polymer | L₃ | L₄ | HL₁-R₁ | HL₂-R₂ |
|---|---|---|---|---|
| B3 | -CH₂-(CH₂)₂-CH2- | >N-(CH₂)₅-OH | NH₂-CH₂-(CH₂)₂-CH(CH₃)-CH₂-NH₂ | H₂N-CH₂-(CH₂)₂-CH(CH₃)-CH₂-NH₂ |
| R3-C32-R3 (R) | -CH₂-(CH₂)₂-CH₂- | >N-(CH₂)₅-OH | H₂N-Arg-Arg-Arg | H₂N-Arg-Arg-Arg |
| K3-C32-K3 | -CH₂-(CH₂)₂-CH₂- | >N-(CH₂)₅-OH | NH₂-Lys-Lys-Lys | H₂N-Lys-Lys-Lys |
| H3-C32-H3 | -CH₂-(CH₂)₂-CH₂- | >N-(CH₂)₅-OH | NH₂-His-His-His | NH₂-His-His-His |
| R3C-C32-CR3 | -CH₂-(CH₂)₂-CH₂- | >N-(CH₂)₅-OH | HS-Cys-Arg-Arg-Arg | HS-Cys-Arg-Arg-Arg |
| K3C-C32-CK3 | -CH₂-(CH₂)₂-CH₂- | >N-(CH₂)₅-OH | HS-Cys-Lys-Lys-Lys | HS-Cys-Lys-Lys-Lys |
| H3C-C32-CH3 | -CH₂-(CH₂)₂-CH₂- | >N-(CH₂)₅-OH | HS-Cys-His-His-His | HS-Cys-His-His-His |
| B3-C32-R3 | -CH₂-(CH₂)₂-CH₂- | >N-(CH₂)₅-OH | H₂N-CH₂-(CH₂)₂-CH(CH₃)-CH₂-NH₂ | H₂N-Arg-Arg-Arg |
| B3-C32-CR3 | -CH₂-(CH₂)₂-CH₂- | >N-(CH₂)₅-OH | H₂N-CH₂-(CH₂)₂-CH(CH₃)-CH₂-NH₂ | HS-Cys-Arg-Arg-Arg |
| B3-C32-CK3 | -CH₂-(CH₂)₂-CH₂- | >N-(CH₂)₅-OH | H₂N-CH₂-(CH₂)₂-CH(CH₃)-CH₂-NH₂ | HS-Cys-Lys-Lys-Lys |
| B3-C32-CH3 | -CH₂-(CH₂)₂-CH₂- | >N-(CH₂)₅-OH | H₂N-CH₂-(CH₂)₂-CH(CH₃)-CH₂-NH₂ | HS-Cys-His-His-His |
| R3C-C32-CK3 | -CH₂-(CH₂)₂-CH₂- | >N-(CH₂)₅-OH | HS-Cys-Arg-Arg-Arg | HS-Cys-Lys-Lys-Lys |
| R3C-C32-CH3 | -CH₂-(CH₂)₂-CH₂- | >N-(CH₂)₅-OH | HS-Cys-Arg-Arg-Arg | HS-Cys-His-His-His |
| K3C-C32-CH3 | -CH₂-(CH₂)₂-CH₂- | >N-(CH₂)₅-OH | HS-Cys-Lys-Lys-Lys | HS-Cys-His-His-His |
| B3-C32SS-B3 | -CH₂-CH₂-S-S-CH₂-CH₂- | >N-(CH₂)₅-OH | H₂N-CH₂-(CH₂)₂-CH(CH₃)-CH₂-NH₂ | H₂N-CH₂-(CH₂)₂-CH(CH₃)-CH₂-NH₂ |
| R3C-C32SS-CR3 | -CH₂-CH₂-S-S-CH₂-CH₂- | >N-(CH₂)₅-OH | HS-Cys-Arg-Arg-Arg | HS-Cys-Arg-Arg-Arg |
| K3C-C32SS-CK3 | -CH₂-CH₂-S-S-CH₂-CH₂- | >N-(CH₂)₅-OH | HS-Cys-Lys-Lys-Lys | HS-Cys-Lys-Lys-Lys |
| H3C-C32SS-CH3 | -CH₂-CH₂-S-S-CH₂-CH₂- | >N-(CH₂)₅-OH | HS-Cys-His-His-His | HS-Cys-His-His-His |
| B3-C32SS-CR3 | -CH₂-CH₂-S-S-CH₂-CH₂- | >N-(CH₂)₅-OH | H₂N-CH₂-(CH₂)₂-CH(CH₃)-CH₂-NH₂ | HS-Cys-Arg-Arg-Arg |
| B3-C32SS-CK3 | -CH₂-CH₂-S-S-CH₂-CH₂- | >N-(CH₂)₅-OH | H₂N-CH₂-(CH₂)₂-CH(CH₃)-CH₂-NH₂ | HS-Cys-Lys-Lys-Lys |
| B3-C32SS-CH3 | -CH₂-CH₂-S-S-CH₂-CH₂- | >N-(CH₂)₅-OH | H₂N-CH₂-(CH₂)₂-CH(CH₃)-CH₂-NH₂ | HS-Cys-His-His-His |
| R3C-C32SS-CK3 | -CH₂-CH₂-S-S-CH₂-CH₂- | >N-(CH₂)₅-OH | HS-Cys-Arg-Arg-Arg | HS-Cys-Lys-Lys-Lys |
| R3C-C32SS-CH3 | -CH₂-CH₂-S-S-CH₂-CH₂- | >N-(CH₂)₅-OH | HS-Cys-Arg-Arg-Arg | HS-Cys-His-His-His |
| K3C-C32SS-CH3 | -CH₂-CH₂-S-S-CH₂-CH₂- | >N-(CH₂)₅-OH | HS-Cys-Lys-Lys-Lys | HS-Cys-His-His-His |
| D3C-C32-CD3 | -CH₂-(CH₂)₂-CH₂- | >N-(CH₂)₅-OH | HS-Cys-Asp-Asp-Asp | HS-Cys-Asp-Asp-Asp |
| E3C-C32-CE3 | -CH₂-(CH₂)₂-CH₂- | >N-(CH₂)₅-OH | HS-Cys-Glu-Glu-Glu | HS-Cys-Glu-Glu-Glu |
| D3C-C32-CE3 | -CH₂-(CH₂)₂-CH₂- | >N-(CH₂)₅-OH | HS-Cys-Asp-Asp-Asp | HS-Cys-Glu-Glu-Glu |
| E3C-C32SS-CD3 | -CH₂-CH₂-S-S-CH₂-CH₂- | >N-(CH₂)₅-OH | HS-Cys-Asp-Asp-Asp | HS-Cys-Asp-Asp-Asp |
| E3C-C32SS-CE3 | -CH₂-CH₂-S-S-CH₂-CH₂- | >N-(CH₂)₅-OH | HS-Cys-Glu-Glu-Glu | HS-Cys-Glu-Glu-Glu |
| D3C-C32SS-CE3 | -CH₂-CH₂-S-S-CH₂-CH₂- | >N-(CH₂)₅-OH | HS-Cys-Asp-Asp-Asp | HS-Cys-Glu-Glu-Glu |

### Example 5: Preparation of MntR, TreR and SucR

In a typical example of the first aspect of the invention, the R polymer was prepared in the presence of mannitol, trehalose or sucrose to form MntR, TreR or SucR, respectively. The procedure was as follows: C32 (75 mg, 0.035 mmol), CR3.4HCl (55 mg, 0.09 mmol) and DMSO (1.5 ml) were placed in Teflon-lined screw cap vials. Then, 10, 20, or 30% (w/w) of the sugar or sugar alcohol were added in each vial and stirred at room temperature for 24 h. The product was purified by precipitation in diethyl ether/acetone (7:3 v/v) for twice and dried under vacuum.

### Example 6: Formation of Complexes of Polymer with DNA

Nanoparticles were formulated by mixing polymer and pGFP (plasmid green fluorescent protein) in a weight ratio of 50:1. For example, pGFP was diluted to 60 μg/ml in a final concentration of ˜5 mM sodium acetate (NaOAc) buffer at pH 5.5 and PBAE stock solution (100 μg/μl) in DMSO was diluted in the same buffer. 100 μl of diluted PBAE solution (3 μg/μl) was added to 100 μl of pGFP, and mixed with vortex for a few seconds and incubated at 37° C. for 30 min.

### Example 7: Further Method for Formation of Complexes of Polymer with DNA

pGFP stock solution was diluted to 60 μg/ml in a final concentration of 11-12 mM of NaAc buffer (pH 5.2). Polymer stock solutions (for example R, MntR, TreR or SucR; 100 mg/ml in DMSO) were diluted in the same buffer. 100 μl of diluted pGFP was added into 100 μl of PBAE solutions (3 mg/ml), and mixed with vortex for a few seconds and incubated at room temperature for 10 min.

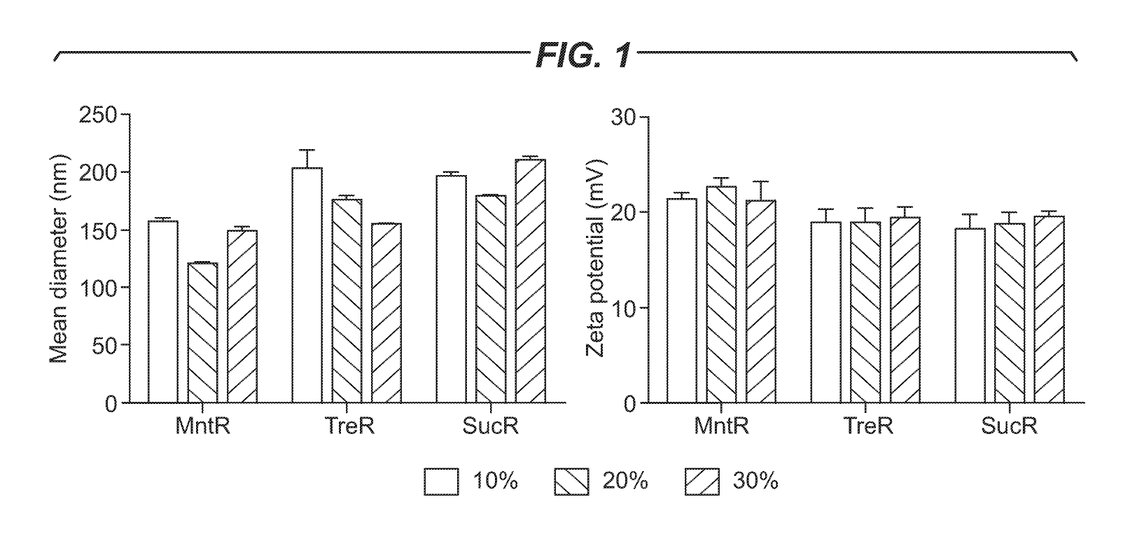

The mean diameter and zeta potential of nanoparticles of pDNA with polymer formed in the presence of 10, 20 or 30% of mannitol, trehalose or sucrose are shown in FIG. 1. MntR polymers were formulated with DNA at a weight ratio of 50:1. MntR/pDNA formed with 20 weight percent mannitol showed the smallest particle size (120.7±1.1 nm) and the highest zeta potential (22.5±1.1 mV). TreR polymers were formulated with DNA at a weight ratio of 100:1, which was general ratio of formulation PBAE/DNA complexes. Increasing the weight percent of trehalose from 10 to 30 decreased the mean size of TreR/pDNA nanoparticles from 202 to 154 nm and negligibly changed their surface charge. SucR:DNA ratio was 50:1. SucR/pDNA formed with 20 weight percent sucrose showed better results than that with 10 or 30 weight percent sucrose. However, the mean size and zeta potential of SucR/pDNA nanoparticles were larger and lower, respectively, than the other sugar-based nanoparticles.

### Example 8: Formation of Complexes of Polymer Plus Chitosan with DNA

20 mg of chitosan was suspended in 0.5% acetic acid (AcOH, 10 ml) and left overnight under stirring at room temperature. The chitosan stock solution (2 mg/ml) was adjusted at pH 5 with 0.1 M NaOH and filtered through 0.22 μm. The chitosan (60 kg/mol and deacetylation degree 60%) stock solution was diluted with 25 mM NaOAc buffer in a proportion ranging from 0.17 to 2.75 weight percent relative to the weight of PBAE when mixed with the PBAE solution. 50 μl of diluted chitosan solution was then added to 50 μl of diluted PBAE solution, and mixed with vortex. 100 μl of GFP (60 μg/ml in the same buffer) was then added to the mixture solution, mixed slightly with vortex and incubated at (a) 37° C. for 30 min or (b) room temperature for 10 min.

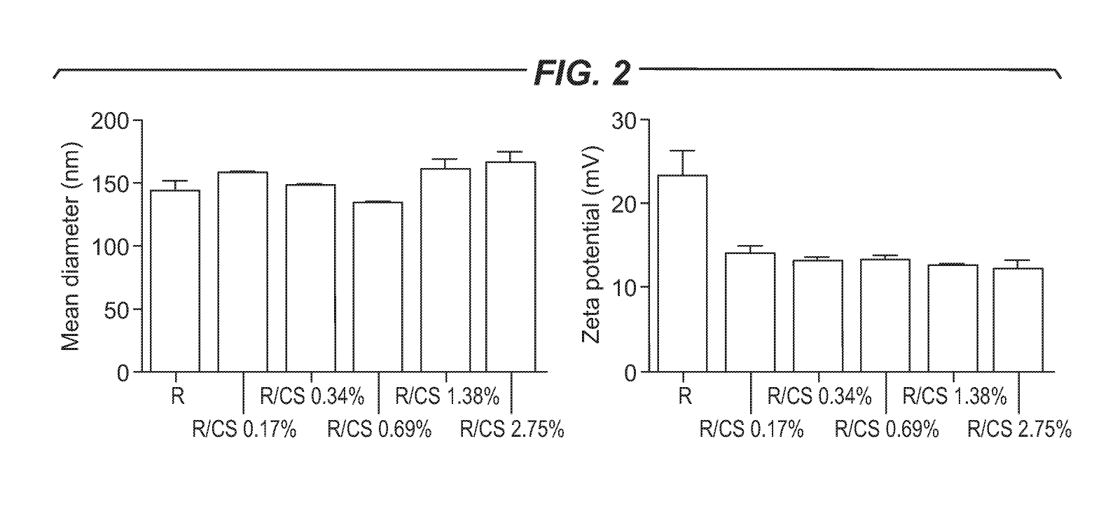

The particle size and zeta potential of R/DNA nanoparticles without or with a coating of 0.17, 0.34, 0.69, 1.38, and 2.75 weight percent chitosan prepared using incubation conditions (a) above were measured and the results are shown in FIG. 2. The mean particle size and zeta potential of R/pDNA as a positive control was 143.2 nm and 23.1 mV, respectively. The R/pDNA nanoparticles coated with chitosan ranged from 126 to 162 nm in the mean diameter and from 12 to 14 mV in zeta potential. There were small differences in size observed between nanoparticles coated with or without chitosan. However, zeta potential values show a significant decrease with the small amount of chitosan. This indicates that chitosan was perfectly formulated on the surface of the complexes.

### Example 9: Formation of Complexes of Polymer Plus Chitosan with DNA

In a separate experiment, further complexes were prepared according to the method of Example 8(b), using chitosan having (a) molecular weight 22 kg/mol, (b) molecular weight 60-120 kg/mol or (c) molecular weight 20-50 kDa in a proportion ranging from 0.17 to 2.67 weight percent relative to the weight of PBAE when mixed with the PBAE solution. Polymers and GFP were in a weight ratio of 50:1.

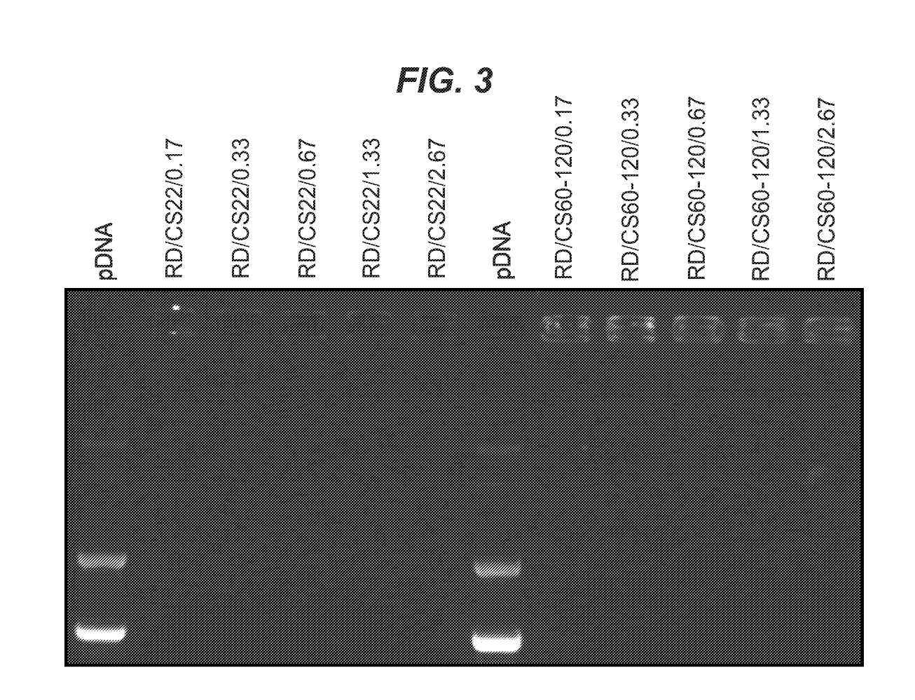

The formation of complexes was confirmed by agarose gel electrophoresis with ethidium bromide, as shown in FIG. 3. In comparison with the mobility of the naked pDNA, the movement of DNA was completely retarded in the test compositions, indicating that addition of chitosan did not affect the formation of PBAE/DNA complexes.

### Example 10: Formation of Sugar-Coated PBAE/DNA Complexes

Complexes of R and pDNA prepared according to the method of Example 7 were coated with 10, 20, 30 and 40 weight percent of mannitol, trehalose or sucrose by incubation at RT for 10 min with a solution containing the relevant amount of mannitol, trehalose or sucrose. Resulting complexes are denoted, for example, R/Mnt10%.

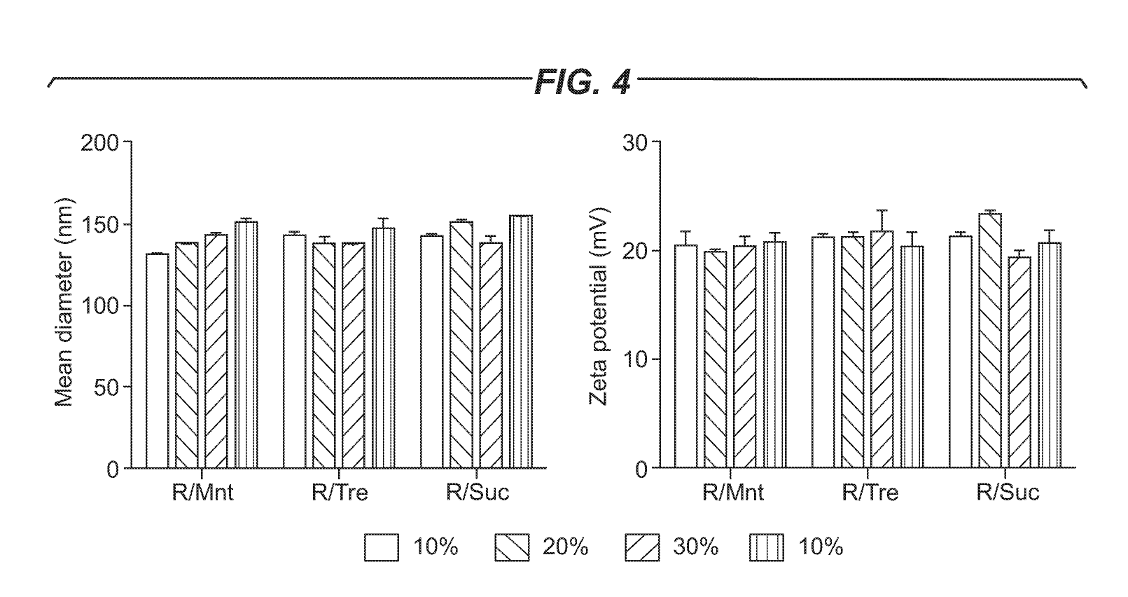

The mean diameter of the resulting sugar-coated nanoparticles was found to be in a range of 130-154 nm with a positive zeta potential of about 19-22 mV, which was slightly decreased compare to the nanoparticles uncoated with sugar (FIG. 4). In the case of nanoparticles coated with mannitol, increasing the amount of mannitol from 10 to 40% increased the mean size from 130 to 150 nm. However, the size of R/pDNA coated with 5% mannitol was larger than that coated with 10% (data not shown). In the case of nanoparticles coated with trehalose or sucrose, the mean particle size and zeta potential of nanoparticles were not dependent on the amount of sugar. R/Mnt10%, 30% R/Tre30%, and R/Suc30% had the smallest diameter.

### Example 11: Factors Affecting Stability

The effect of different factors on the stability of nanoparticles was investigated. (i) Preparation according to Example 6 (i.e. incubation at 37° C. for 30 min) with a final concentration of ˜5 mM sodium acetate; (ii) preparation according to Example 6 but with a final concentration of 11-12 mM sodium acetate and (iii) Example 7 (i.e. incubation at RT for 10 min) with a final concentration of 11-12 mM sodium acetate. The stability of the nanoparticles, diluted in PBS to a final concentration of 0.25 mg/ml, was determined. While PBAE stock solution can be obtained at a constant concentration of 100 mg/ml, DNA stock solutions can be obtained at various concentrations and this may influence the various size and zeta potential of complexes.

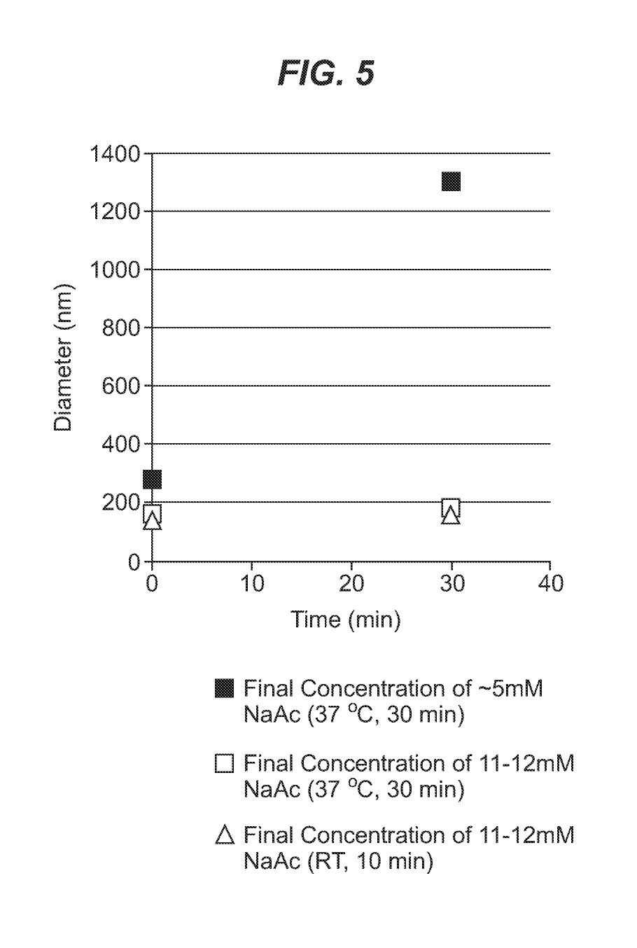

The results are shown in FIG. 5 (indicated values are mean±SD of at least three experiments). The complexes at a final concentration of 11-12 mM of sodium acetate buffer were slightly smaller and much more stable than those in 5 mM of sodium acetate buffer. These results suggest that the final concentration of sodium acetate buffer affects the ionic strength of the complexes, and preparation of nanoparticles in the presence of buffers at 11-12 mM significantly increases stability. Further, the duration and temperature of incubation did not have an effect on the size or the stability of the nanoparticles over the 30 min measurement time frame. Accordingly, it is noted that the stability of complexes are not affected by the incubation conditions, but by the concentration of sodium acetate buffer (ionic strength).

### Example 12: Stability of Particles Coated with Chitosan

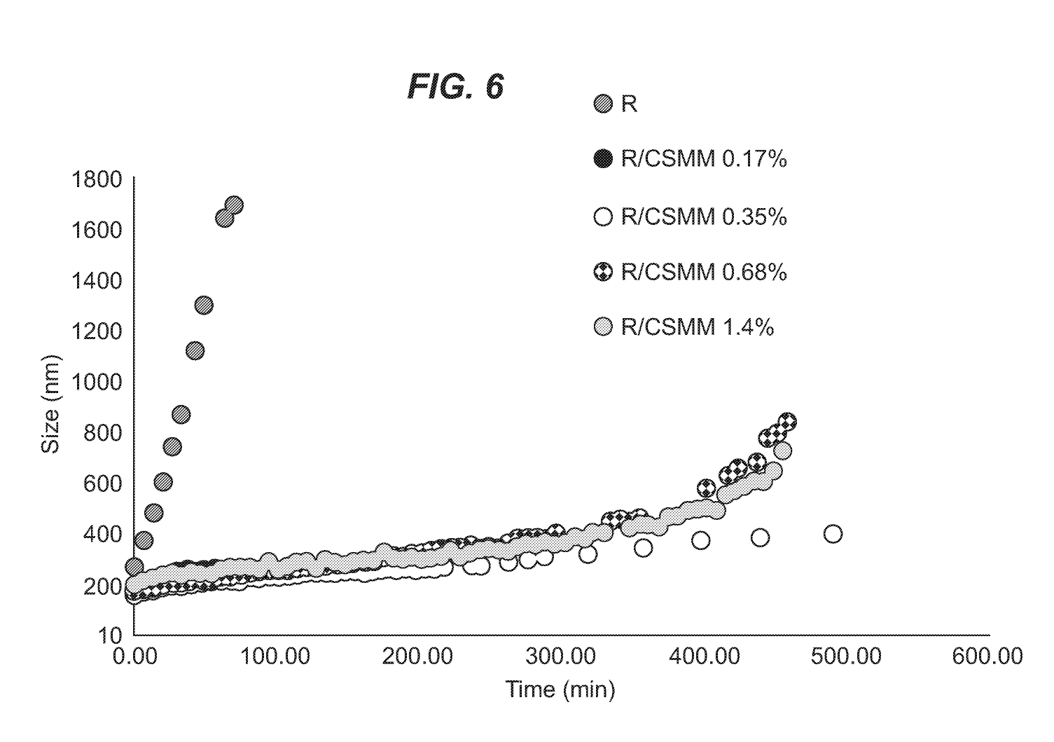

Nanoparticles comprising polymer R in combination with differing amounts of chitosan prepared according to Example 8(a) were incubated for 8 h in phosphate-buffered saline and were analysed by DLS every 5 min in order to monitor changes in the size of the nanoparticles. The results are shown in FIG. 6. Nanoparticles having 0.17 or 0.35 weight percent chitosan were seen to be particularly resistant to agglomeration.

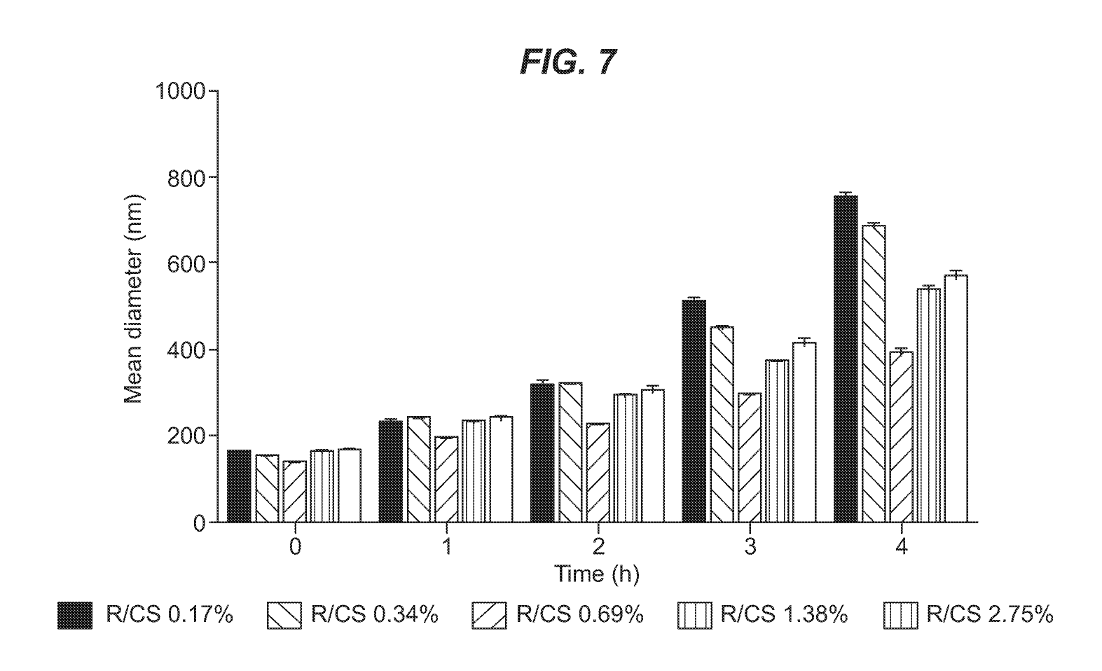

The stability over 4 h of R/DNA nanoparticles prepared according to Example 8(b) with a coating of 0.17, 0.34, 0.69, 1.38, and 2.75 weight percent chitosan was measured. The results are shown in FIG. 7. Notably, the mean particle size of R/pDNA coated with 0.69% of chitosan was still less than 400 nm at 4 h, indicating that 0.69 weight percent of chitosan may be an optimal content to sustain the formulation of the nanoparticles.

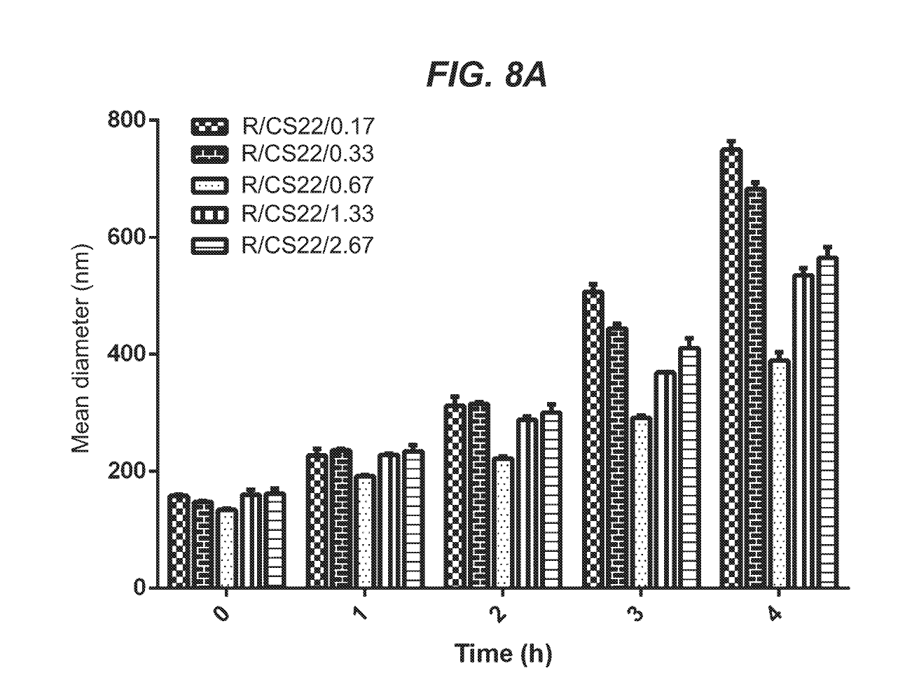

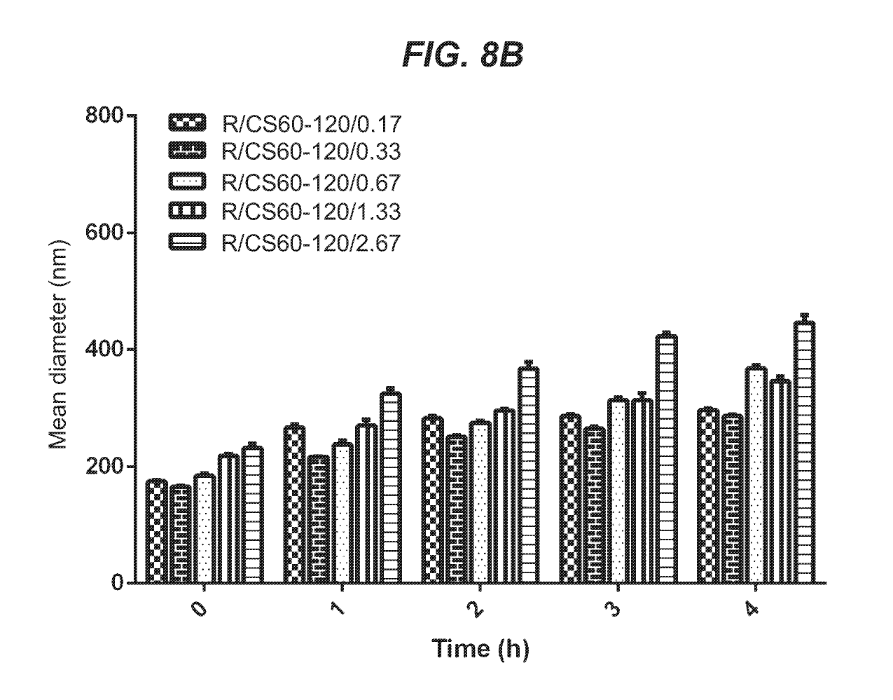

The stability over 4 h of R/DNA nanoparticles prepared according to Example 9(a) and Example 9(b) with a coating of 0.17, 0.33, 0.67, 1.33, and 2.67 weight percent chitosan was measured. The results are shown in FIGS. 8(a) and 8(b) respectively.

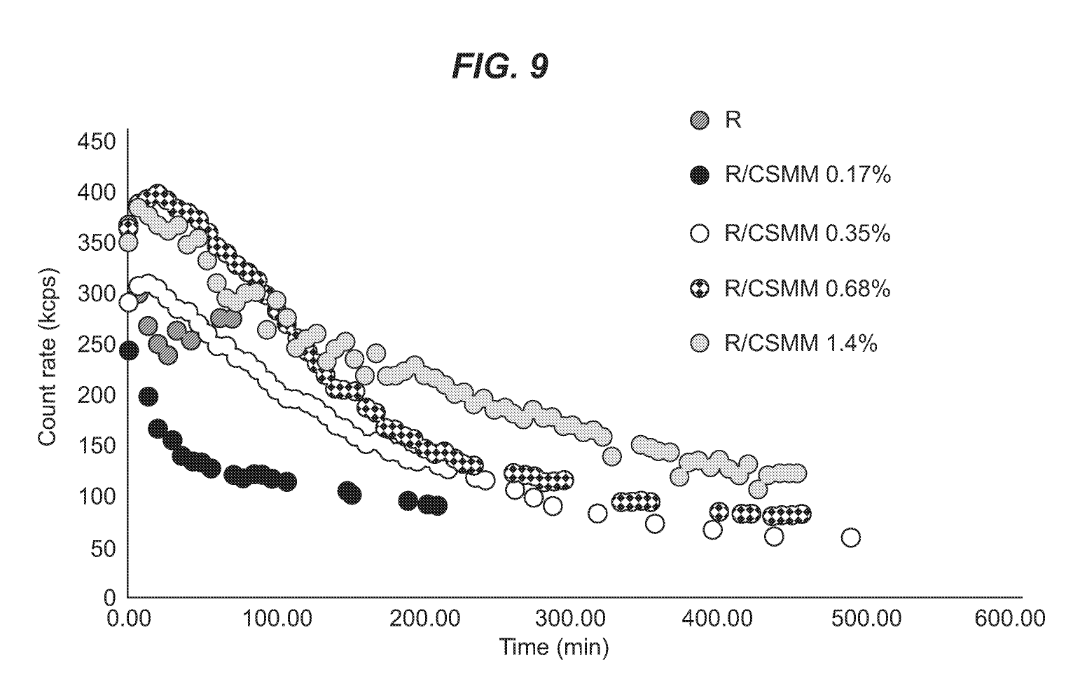

NRK-52e cells using nanoparticles comprising R and differing amounts of chitosan prepared according to Example 8(a) as carriers of pGFP at 0.6 μg/well were also analysed by fluorescence microscopy every 5 min over 8 h in order to monitor changes in the degradation profile of the nanoparticles over time. The results are shown in FIG. 9. Nanoparticles with higher amounts of chitosan showed a slower decrease in the count rate, suggesting that they were resistant to degradation.

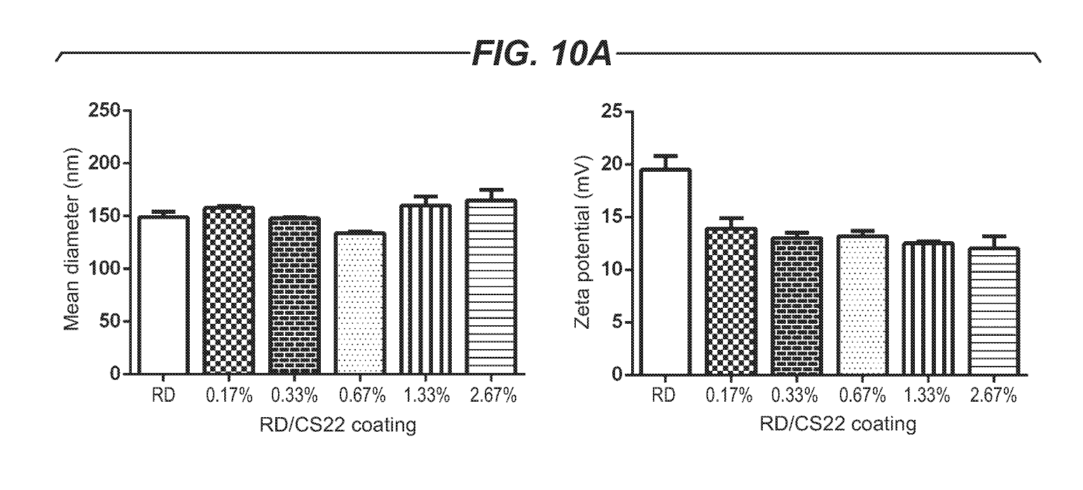

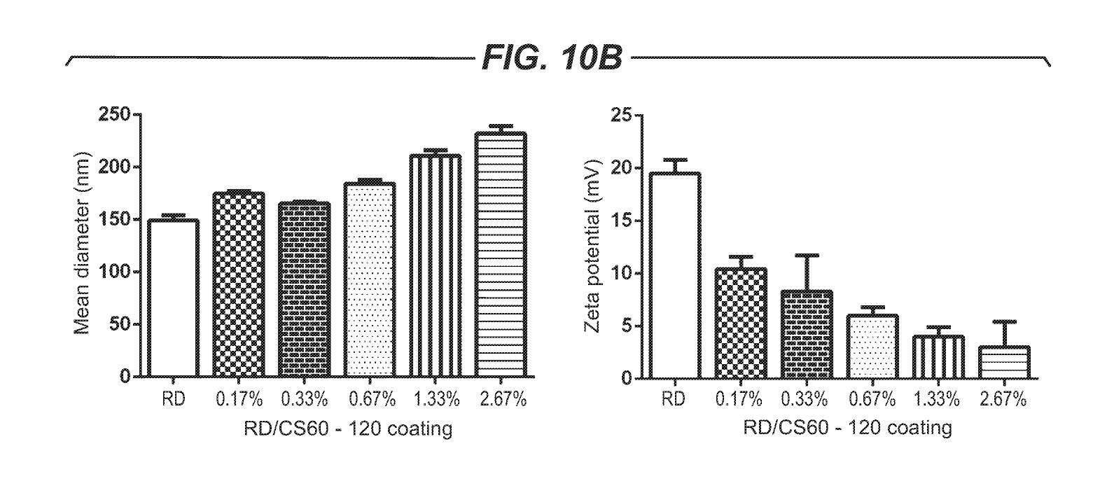

The stability over 4 h of R/DNA nanoparticles prepared according to Example 9 with a coating of 0.17, 0.33, 0.67, 1.33, and 2.67 weight percent chitosan was measured. The results are shown in FIG. 10.

### Example 13: Stability of Nanoparticles Coated with Mannitol

R/Mnt complexes prepared according to Example 10 were analysed by DLS every hour in order to monitor changes in the diameter of the nanoparticles.

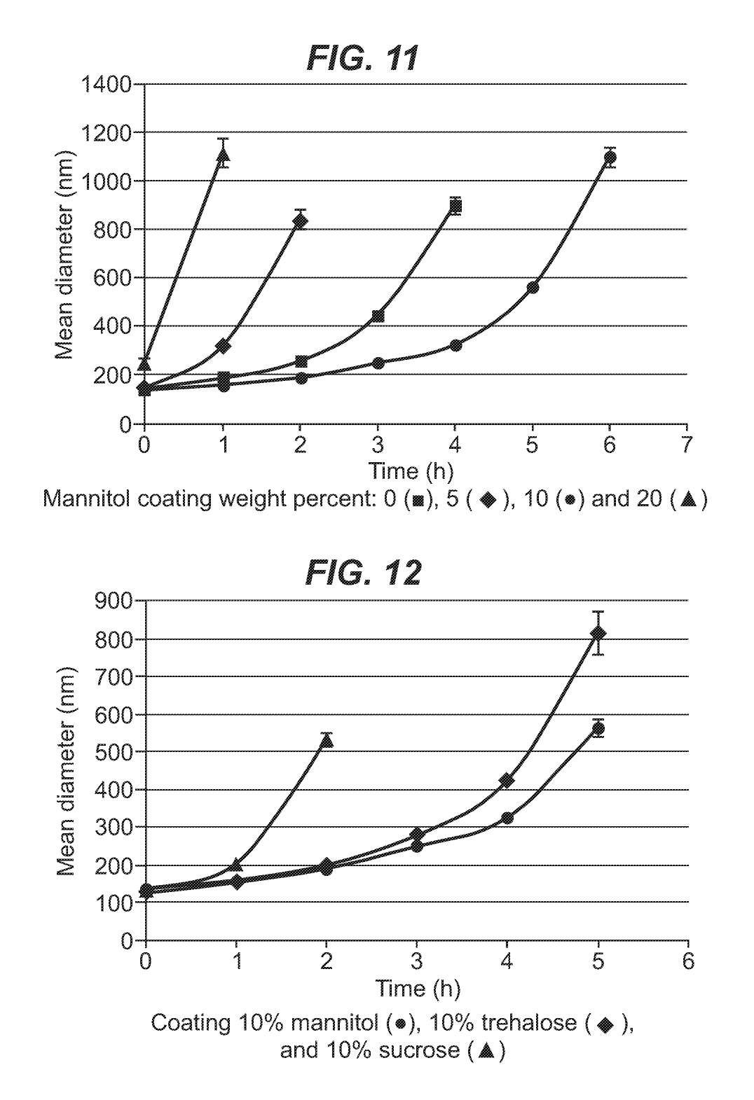

The results are shown in FIG. 11 (indicated values are mean±SD of at least three experiments). The mean diameter for each type of nanoparticles is in the range 140-150 nm at t=0 and increases at each hourly measurement. It can be seen that nanoparticles coated with 10 weight percent mannitol are more stable than those without mannitol, whereas those coated with 5 or 20 weight percent mannitol are less stable than those without mannitol.

### Example 14: Stability of Nanoparticles Coated with Mannitol, Sucrose or Trehalose

Complexes coated with 10 weight percent mannitol, sucrose or trehalose prepared according to Example 10 were analysed by DLS every hour in order to monitor changes in the diameter of the nanoparticles. The results are shown in FIG. 12 (indicated values are mean±SD of at least three experiments). It can be seen that nanoparticles coated with 10 weight percent mannitol are more stable than those coated with 10 weight percent sucrose or 10 weight percent trehalose.

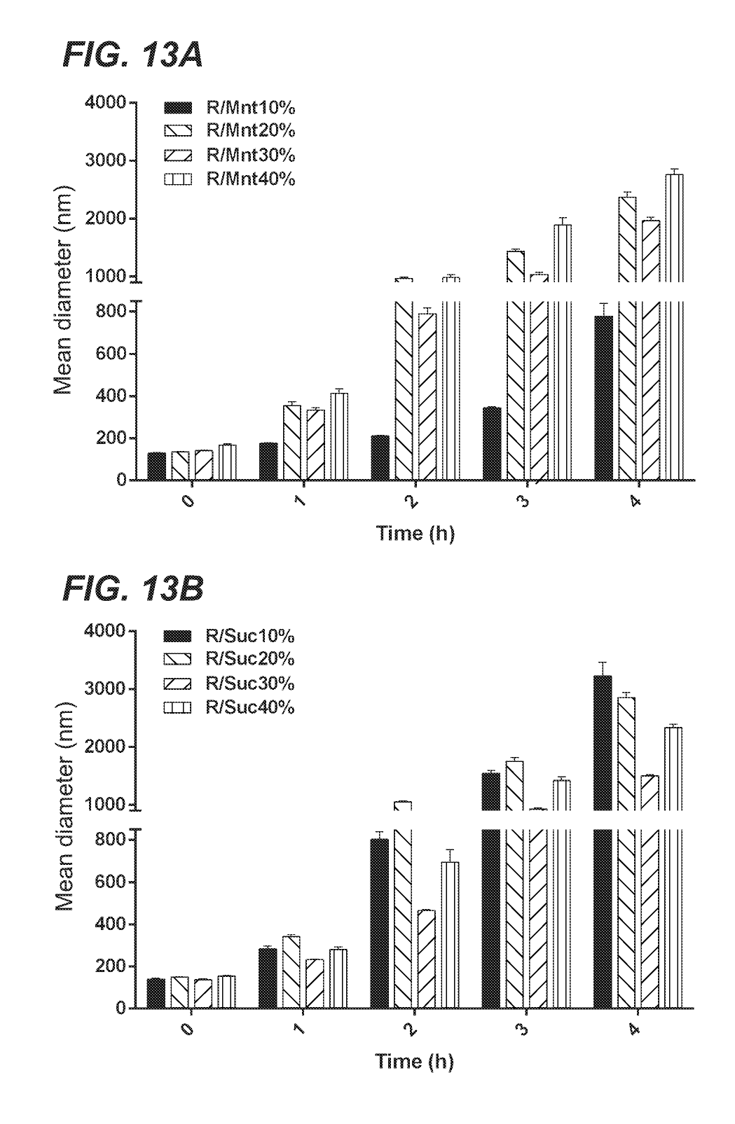

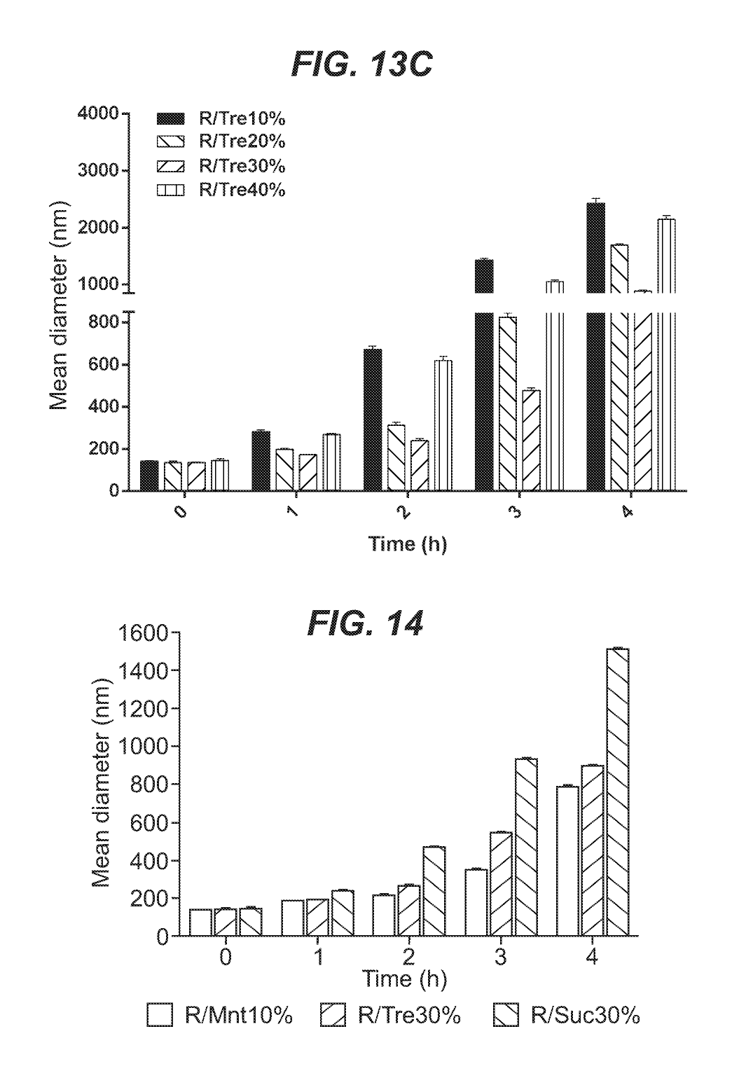

In a separate experiment, nanoparticles coated with 10 to 40% of mannitol, trehalose or sucrose were incubated for 4 h in PBS at pH7.4 and analysed by DLS every hour in order to observe changes in the size of complexes. The results are shown in FIG. 13 (indicated values are mean±SD of at least three experiments). There highest stability for each coating material was observed with 10% of mannitol (R/Mnt10%), 30% of both sucrose (R/Suc30%) and trehalose (R/Tre30%).

The results for R/Mnt10%, R/Tre30% and R/Suc30% are shown in FIG. 14. The mean size of both R/Mnt10% and R/Tre30% was less than 800 nm within 4h. On the other hand, the size of R/Suc30% rapidly increased compare to that of the others. These results support that the stability of nanoparticles was dependent on the amount of sugar, and 10 weight percent of mannitol, or 30 weight percent of trehalose or sucrose are optimal weight contents for improving the stability of complexes. The zeta potential of all nanoparticles slightly decreased within 4 h (data not shown).

### Example 15: Stability of Nanoparticles Formed from MntR or with Mannitol Coating

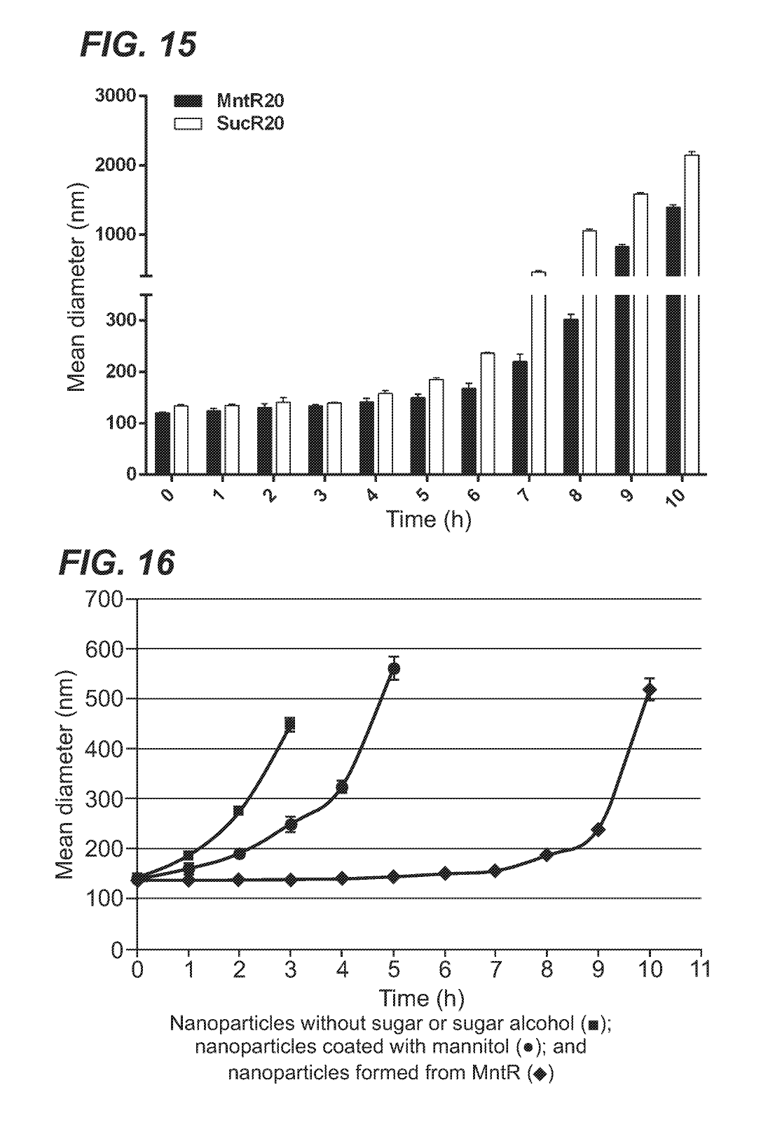

The stability over 10 h of complexes of MntR20 and DNA and of SucR20 in PBS at pH7.4 was measured by DLS every hour. The results are shown in FIG. 15 (indicated values are mean±SD of at least three experiments). There was minimal increase in the particle size of MntR20 and SucR20 within 4 h in PBS. The particle size of the complexes increased with decreasing zeta potential (data not shown). All results indicate that the presence of additives during polymerization results in complexes having high stability.

### Example 16: Stability of Nanoparticles Formed from MntR or with Mannitol Coating

Nanoparticles comprising pGFP were prepared from R polymer according to Example 7 either (i) without mannitol, (ii) with coating of mannitol at 10 weight percent, (iii) polymerized with mannitol at 10 weight percent. The properties of these nanoparticles are shown in Table 2. There were no significant differences between the zeta potentials of these nanoparticles.

TABLE 2
Properties of pGFP nanoparticle

| Nanoparticle | Mean diameter | | zeta potential |
|---|---|---|---|
| preparation | (nm) | Polydispersity | (mV) |
| (i) R | 143.2 ± 2.4 | 0.184 ± 0.044 | 23 ± 3.1 |
| (ii) R/Mnt10 | 139.7 ± 2.0 | 0.198 ± 0.014 | 24.1 ± 2.5 |
| (iii) MntR10 | 137.6 ± 2.4 | 0.135 ± 0.025 | 24 ± 3.0 |

The nanoparticles were diluted in phosphate buffer pH 7.4 and analysed by DLS every hour in order to monitor changes in the diameter of the nanoparticles. The results are shown in FIG. 16 (indicated values are mean±SD of at least three experiments). As expected from Example 14, the nanoparticles coated with 10 weight percent mannitol were more stable (less prone to aggregation) than the nanoparticles without mannitol. The nanoparticles having 10 weight percent mannitol added prior to the polymerization step are significantly more stable even than those coated with 10 weight percent mannitol: after 8 hours the mean diameter was still <200 nm.

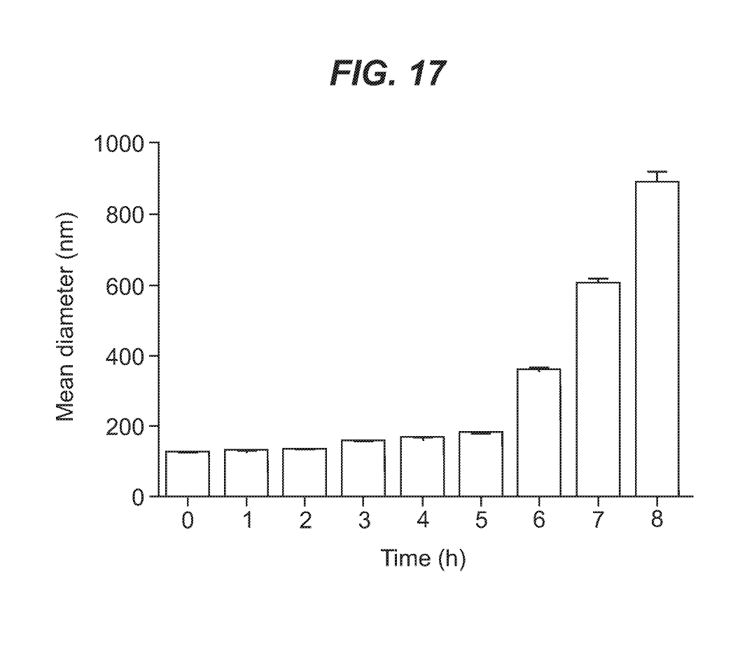

The stability of MntR20% nanoparticles over time was also measured. The results are shown in FIG. 17. The mean diameter of MntR20/pDNA was 600.4±17.6 nm within 7 h, indicating that MntR20/pDNA nanoparticles significantly increased the stability of the nanoparticles. The particle size of nanoparticles increased with decreasing zeta potential, indicating the weak binding between DNA and polymers (data not shown).

### Example 17: Effect of Chitosan on Transfection Efficiency in NRK-52e Cells

NRK-52e cells were seeded in 96-well plates at 10,000 cells/well and incubated overnight to roughly 80% confluence. The cells were then transfected with the following:
Nanoparticles comprising R
Nanoparticles comprising R and coatings of differing amounts of chitosan
Chitosan alone
GeneJuice (positive control)
No treatment (negative control)

In each case GFP expression was evaluated 48 h after transfection by fluorescence microscopy and quantified by flow cytometry based on a sample of 2000-5000 cells. The properties of transfected cells that were measured were relative size, relative granularity or internal complexity, and relative fluorescence intensity. Finally, data were analysed by BD LSRFortessa cell analyser software.

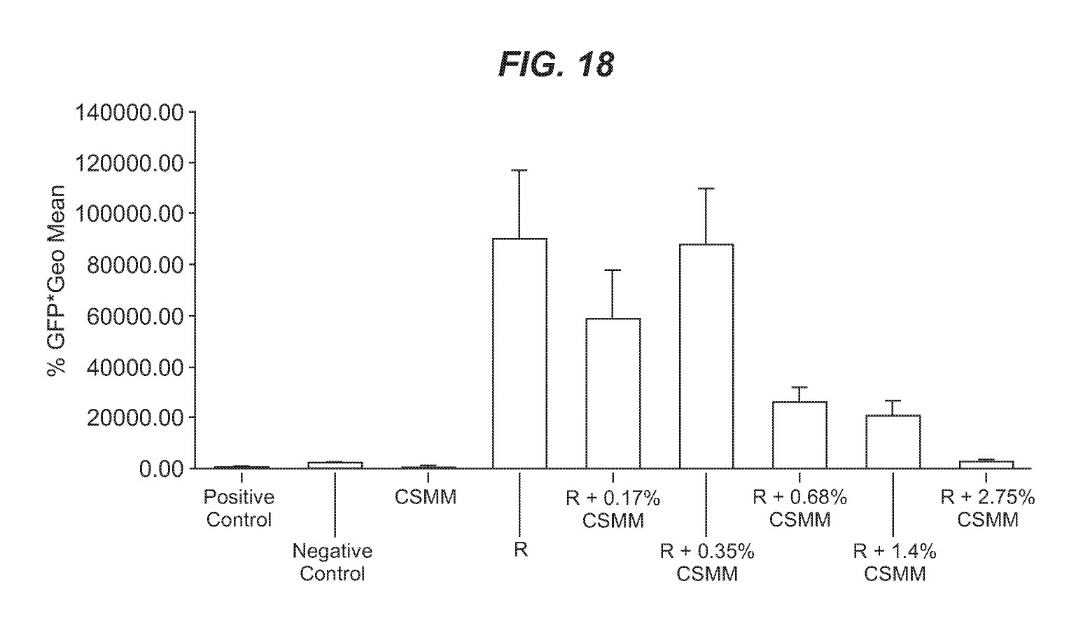

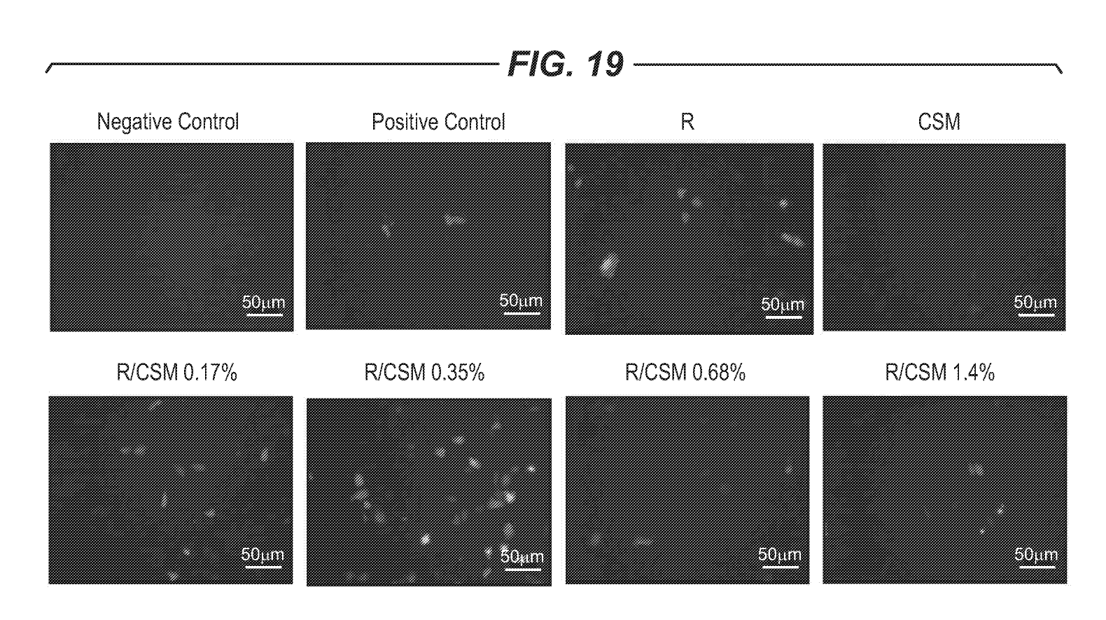

Results of flow cytometry are shown in FIG. 18 and results of fluorescence microscopy are shown in FIG. 19. It can be seen that all the polymer formulations achieved higher transfection efficiency than positive control and chitosan alone. Further, when the weight percent of chitosan was increased the transfection efficiency was at a level similar to that of the polymer alone, but with further increase in the weight percent of chitosan, transfection efficiency was reduced. In particular, chitosan was found to result in the highest transfection efficiency when present at 0.17 to 0.35 weight percent.

The effect of the choice of end modification of the PBAE was also investigated. The following nanoparticles were prepared and tested according to the protocol above:
Nanoparticles comprising R.
Nanoparticles comprising R and H3C-C32-CH3 at 1:1 weight ratio, with or without inclusion of chitosan at 0.35 weight percent.
Nanoparticles comprising R and D3C-C32-CD3 at 7:3 weight ratio (use of oppositely charged polymers results in increased stability as a result of electrostatic interactions), with or without inclusion of chitosan at 0.35 weight percent.
Chitosan alone.
GeneJuice (positive control).
No treatment (negative control).

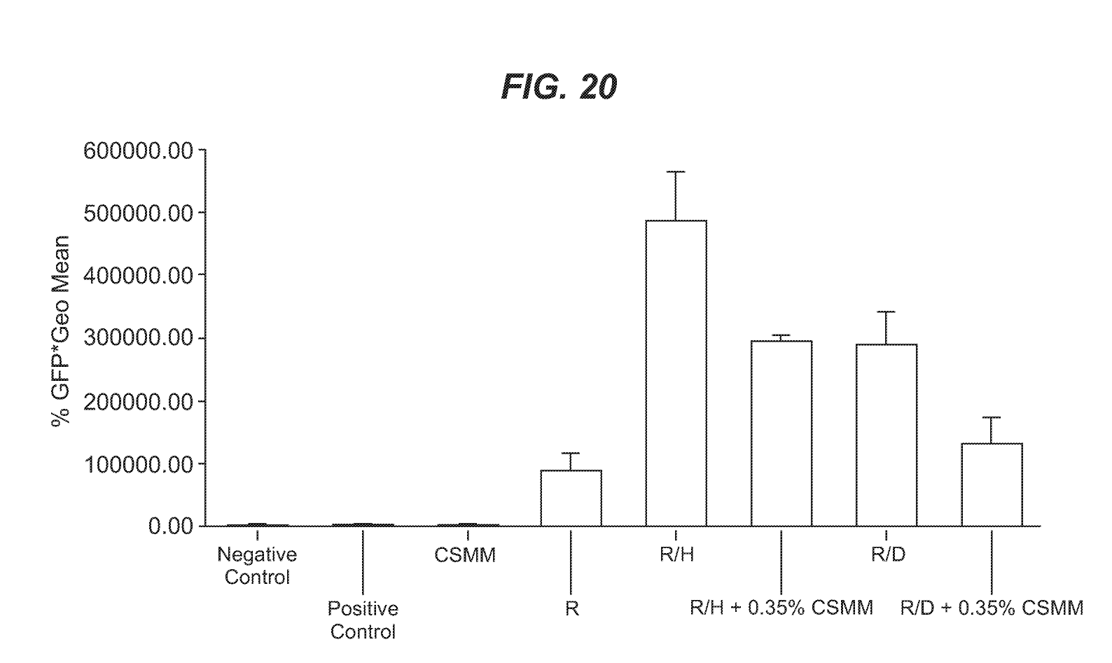

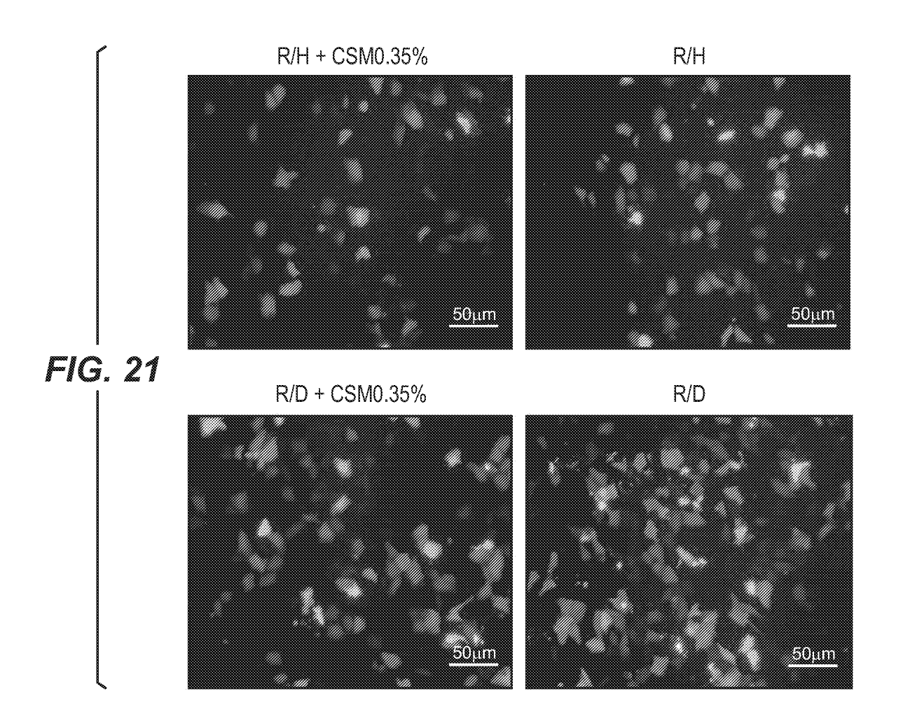

Results of flow cytometry are shown in FIG. 20 and results of fluorescence microscopy are shown in FIG. 21. The lower GFP expression after transfection with nanoparticles comprising chitosan, in spite of transfection levels in the same range, seems to be a result of the increased stability of these nanoparticles, which leads to decreased release of pGFP within transfected cells.

### Example 18: Effect of Chitosan on Transfection Efficiency in COS-7 Cells

COS-7 cells were transfected with pGFP plasmid DNA using nanoparticles prepared according to Example 9(b) and Example 9(c). Non-coated R complexes were used as a positive control to compare the influence of chitosan on the transfection efficiency because R/DNA complexes were reported to provide higher gene expression in cell-type-specific manner and better cellular viability compared to other end-modified PBAEs and commercial transfection agents (Segovia et al., 2014). Cells without any treatment were included as a negative control (NC)

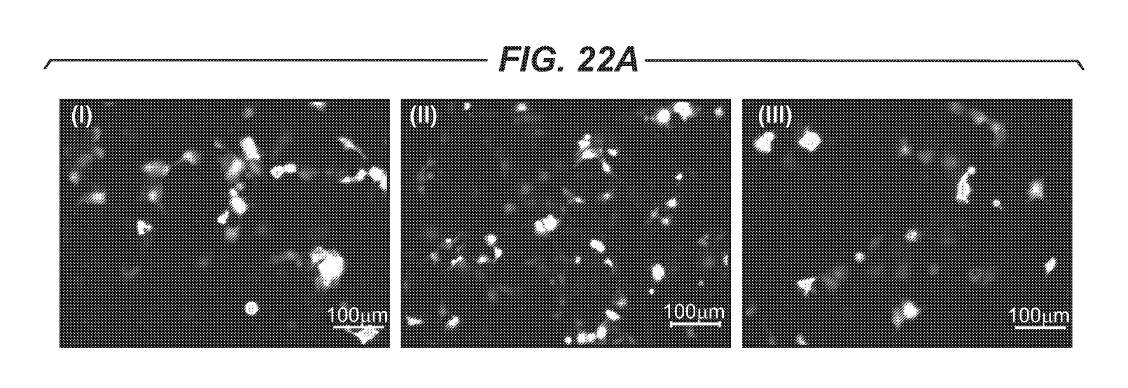

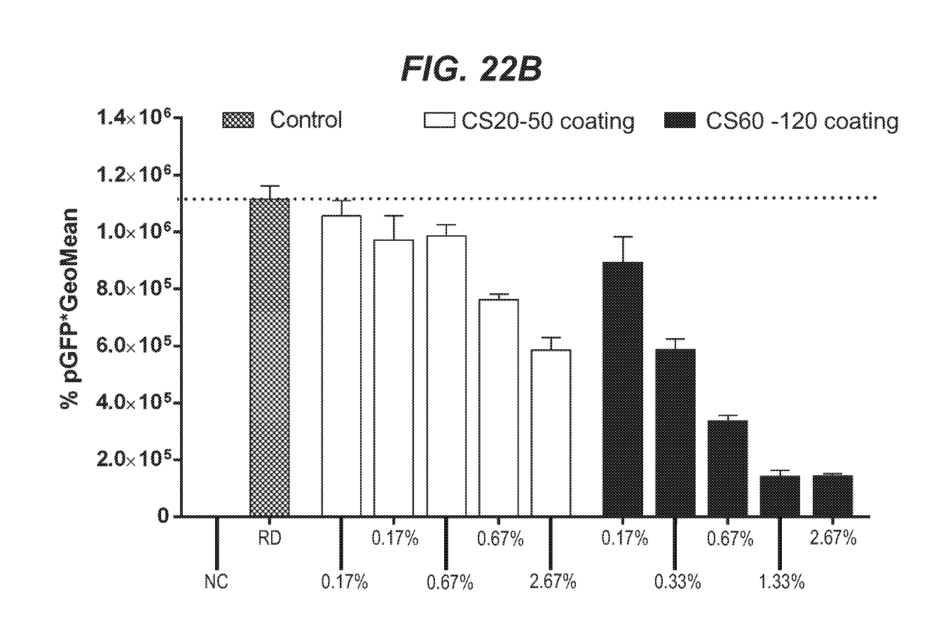

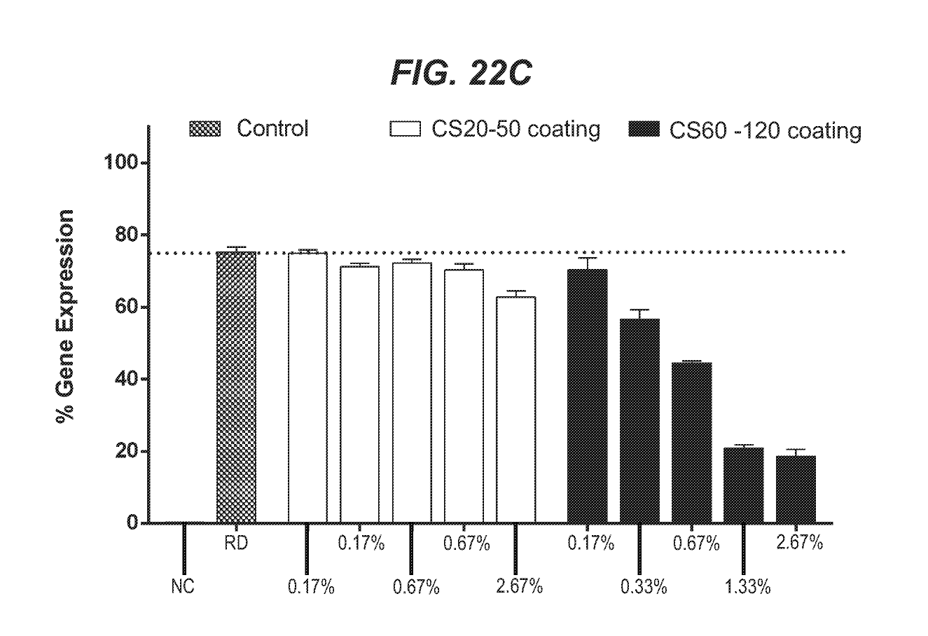

The pGFP expression was determined by flow cytometry analysis at 48 h post-transfection. Results of flow cytometry and fluorescence microscopy are illustrated in FIG. 22 (indicated values in b and c are mean±SD of at least three experiments). In FIG. 22c, GFP expression was determined after 48 h by flow cytometry and bars represent percentage of cells positively transfected and the normalized total gene expression.

In the case of a coating of chitosan 60-120 kDa, the transfection efficiency of R/DNA coated with chitosan 60-120 kDa decreased with increasing the amount of CSM. The lower GFP expression after transfection with complexes comprising CSM may be a result of the increased size and the decreased zeta potential of these complexes, leading to reduced release of GFP within transfected cells.

Although GFP expression levels slightly decreased with increasing the amount of a coating of chitosan 20-50 kDa, all such complexes except that coated with 2.67% chitosan showed high transfection efficiency (≧75%). Interestingly, higher gene expression was maintained for complexes coated with 0.67 wt % of chitosan 20-50 kDa, which also showed the smallest size and the highest stability among the chitosan coated complexes.

### Example 19: Effect of Sugar, Sugar Alcohol or Chitosan on In Vitro Transfection

Cellular transfection was carried out using pDNA plasmid in COS-7 cells. Cells were seeded on 96-well plated at 10,000 cells/well and incubated overnight prior to performing the transfection experiments. PBAE:pDNA complexes were prepared as described above (PBAE:DNA=wt:wt, 50:1). Complexes were diluted in serum-free DMEM medium and added to cells at a final plasmid concentration of 0.3 μg pDNA/well. Briefly, 33 μl of PBAE/DNA complexes were diluted into 450 μl of serum-free DMEM medium and cells were washed once with PBS. Then, 150 μl of the resulting solutions were added to each well, achieving a final concentration of 0.3 μg pDNA/well. Cells were incubated for 3 h at 37° C. in 5% CO₂ atmosphere. Subsequently, cells were washed once with PBS, and complete DMEM medium was added. Cells were harvested after 48 h and analysed for GFP expression by flow cytometry (BD LSRFortessa cell analyzer). GFP expression was compared against a negative control (untreated cells), and both GeneJuice (Merck KGaA, Germany) and unmodified R/pDNA as a positive control.

Transfection conditions for GeneJuice control were optimized in order to achieve maximal transfection efficiency. Different GeneJuice:pGFP ratios were evaluated and 3:1 ratio (v:w) was found to be optimal. Briefly, 300 μl serum-free DMEM medium and 2.88 μl of GeneJuice were mixed vigorously with vortex and incubated at room temperature for 5 min. Then, 15 μl of a plasmid solution at a concentration of 0.06 μg/μl in NaAc buffer was added into the mixture, mixed gently by pipetting and incubated at room temperature for 15 min. Finally, 100 μl of the resulting solution was added to each well, achieving a final concentration of 0.3 μg/well DNA dose.

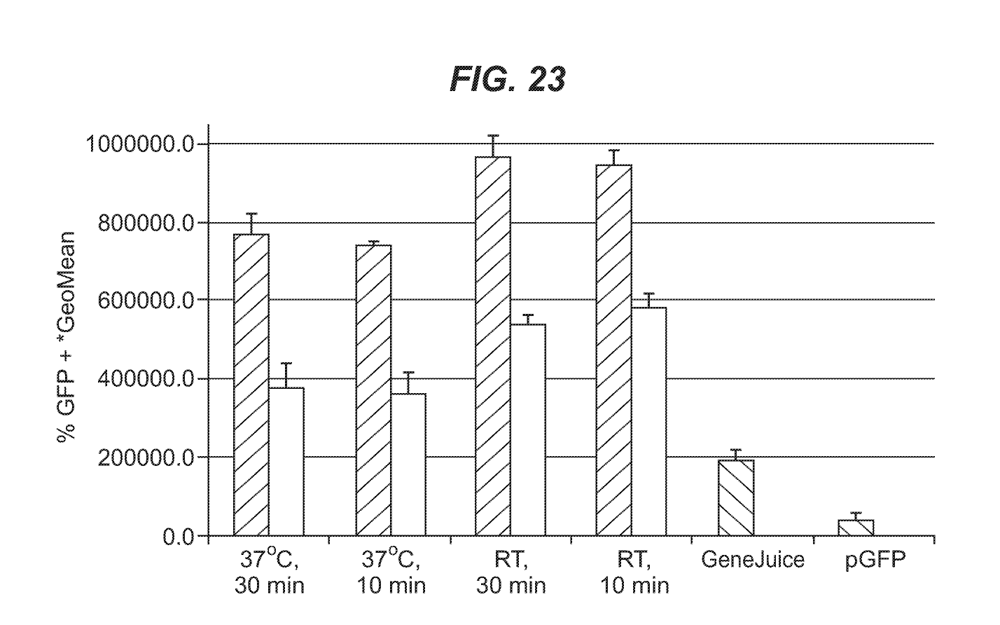

The influence of incubation conditions on transfection efficiency was investigated using MntR20/pDNA, which was shown above to have the highest stability, and R/pDNA obtained under different incubation conditions as shown in FIG. 23. The transfection efficiency of R/pDNA significantly increased when nanoparticles were prepared at RT. However, incubation time resulted in a negligible change in transfection efficiency.

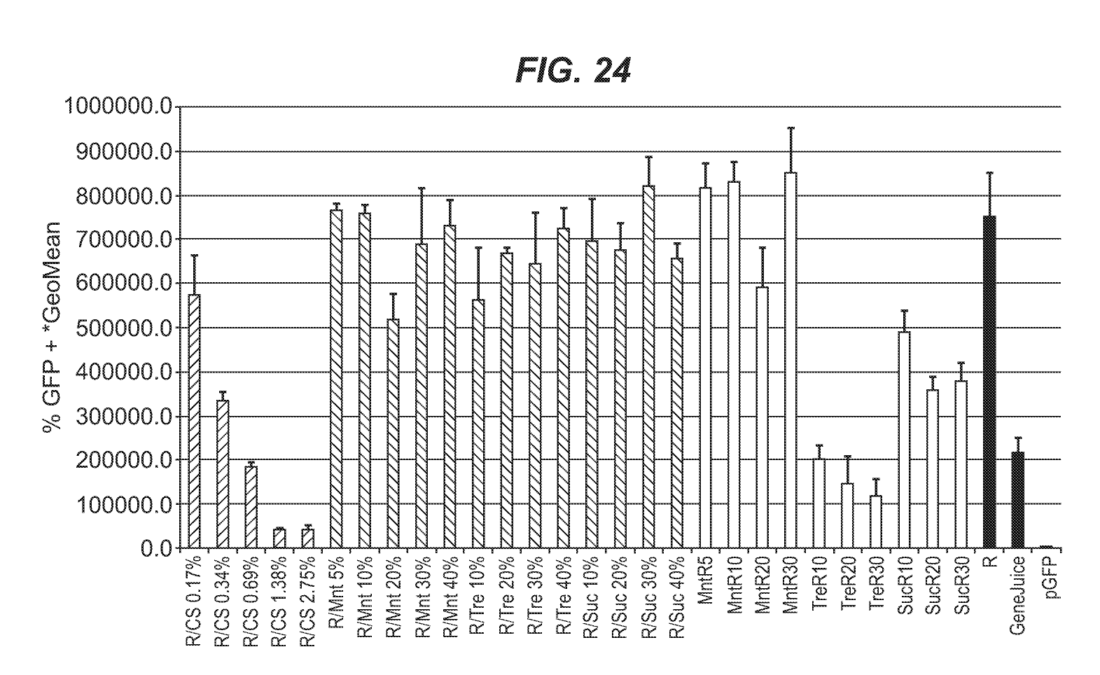

The transfection efficiencies of the nanoparticles modified with chitosan, sugar or sugar alcohol as described in the examples above were measured and the results, in comparison with nanoparticles formed from R, GeneJuice (positive control) and pGFP alone are shown in FIG. 24.

In the case of R/pDNA coated with chitosan, the transfection efficiency decreased as the amount of chitosan was increased. R/pDNA coated with 0.69 weight percent chitosan, which showed the highest stability among those with chitosan, exhibited similar transfection efficiency compared to GeneJuice. However, the transfection efficiency of R/pDNA coated with 1.38 and 2.75 weight percent of chitosan was significantly reduced. This is believed to be because the transfection efficiency of nanoparticles formulated with chitosan is dependent on the pH of the medium due to its pKa, and efficiency is dramatically decreased at a transfection medium pH of 7.4 (Mao 2010).

Overall high transfection efficiency was obtained from R/pDNA coated with sugar, especially with mannitol. R/pDNA coated with 5 and 10 weight percent of mannitol or 30 weight percent sucrose showed higher transfection efficiency compared with uncoated nanoparticles. The lowest expression efficiencies were seen using 20 weight percent mannitol and 10 weight percent trehalose. These results confirm that the transfection efficiency of nanoparticles is dependent on either weight percent or type of sugar, and mannitol can promote higher both stability and transfection of nanoparticles than those of trehalose or sucrose.

MntR nanoparticles showed much higher transfection efficiency than coated nanoparticles. Moreover, MntR showed higher expression efficiency than TreR or SucR. Surprisingly, MntR20 significantly decreased the transfection efficiency. This corresponds to the results obtained from nanoparticles coated with 20 weight percent mannitol.

Very low transfection efficiency was observed with the TreR-based nanoparticles, which may be a result of the lower plasmid dosage of 0.075 μg/well (0.3 μg/well) that was used to avoid cytotoxicity resulting from the high TreR:DNA ratio of 100:1 that was needed for formulated nanoparticles.

The lowest transfection efficiency was detected for MntR20, TreR30, and SucR20, which showed the best formulation and the highest stability among their series. These results are in agreement with the results obtained by Vuorimaa et al., who reported that for tight DNA binders further increase in binding affinity is expected to decrease transfection due to the impaired DNA release in the cells.

### Example 20: Effect of Complexes on Cell Viability

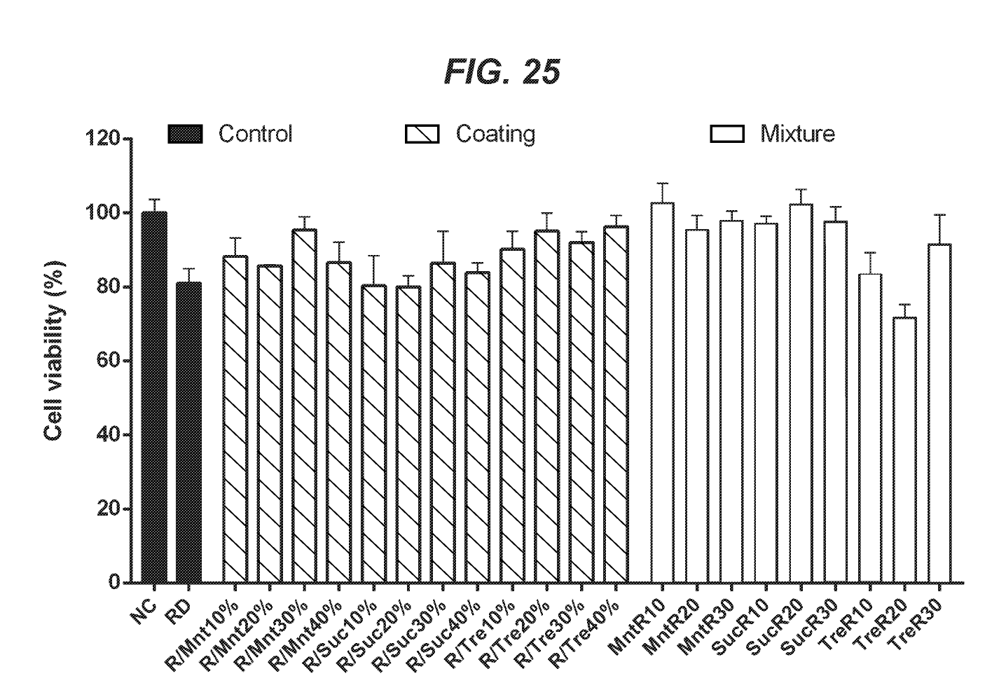

Sugar and sugar alcohol modified complexes were tested for their effect on cell viability of transfected cells. Complexes were formulated at a 50:1 polymer:DNA weight ratio and tested using a plasmid dosage of 0.3 μg/well, except TreR/DNA complexes which were formulated at a 100:1 weight ratio of polymer to DNA was tested using a 0.075 μg DNA/well. As shown in FIG. 25, no large decrease in cell viability was observed for any of the modified complexes compared with the unmodified R complexes, which presented cell viability of ˜81%. These results indicate a good safety profile for the sugar or sugar alcohol modified PBAE/DNA complexes.

1. A nanoparticle comprising an end-modified poly(β-amino ester) and 1 to 35 weight percent, relative to the end-modified poly(β-amino ester), of a sugar or sugar alcohol.

2. A nanoparticle according to claim 1, comprising 2 to 15 weight percent, relative to the end-modified poly(β-amino ester), of the sugar or sugar alcohol.

3. A nanoparticle according to claim 1 wherein the nanoparticle has a coating comprising a sugar or sugar alcohol.

4. A nanoparticle according to claim 3, comprising 1 to 18 weight percent, relative to the end-modified poly(β-amino ester), of the sugar or sugar alcohol.

5. A nanoparticle according to any one of claims 1 to 4, wherein the sugar or sugar alcohol is a sugar alcohol having the general formula HOCH₂(CHOH)ₙCH₂OH wherein n is from 3 to 4

6. A nanoparticle according to claim 5, wherein the sugar alcohol is mannitol.

7. A nanoparticle comprising an end-modified poly(β-amino ester) and chitosan, or a pharmaceutically acceptable salt thereof, at 0.15 to 3.0 weight percent relative to the end-modified poly(β-amino ester).

8. A nanoparticle according to any preceding claim, wherein each end modification of the end-modified poly(β-amino ester) is independently selected from an oligopeptide and R_{y}; wherein R_{y} is selected from the group consisting of hydrogen, halogen, alkyl, cycloalkyl, alkenyl, cycloalkenyl, heteroalkyl, heterocycloalkyl, acyl, aryl or heteroaryl.

9. A nanoparticle according to any preceding claim, wherein the end-modified poly(β-amino ester) is a polymer of formula I: 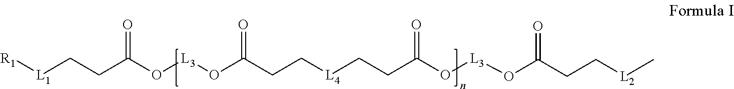
wherein
each L₁ and L₂ is independently selected from the group consisting of 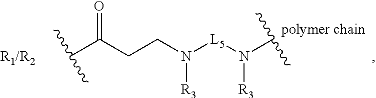
O, S, NRₓ and a bond; wherein Rₓ is independently selected from the group consisting of hydrogen, halogen, alkyl, cycloalkyl, alkenyl, cycloalkenyl, heteroalkyl, heterocycloalkyl, acyl, aryl or heteroaryl;
L₃ is independently selected from the group consisting of alkylene, alkenylene, heteroalkylene, heteroalkenylene, arylene, heteroarylene and 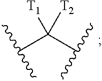
wherein T₁ is 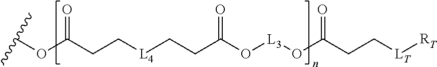
and
T₂ is selected from H, alkyl or 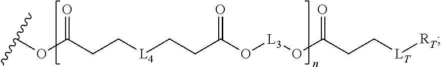
wherein L_{T} is independently selected from the group consisting of: 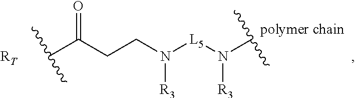
O, S, NRₓ and a bond; wherein Rₓ is independently selected from the group consisting of hydrogen, halogen, alkyl, cycloalkyl, alkenyl, cycloalkenyl, heteroalkyl, heterocycloalkyl, acyl, aryl or heteroaryl;
L₄ is selected from the group consisting of 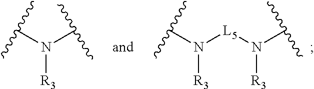
L₅ is independently selected from the group consisting of alkylene, alkenylene, heteroalkylene, heteroalkenylene, arylene or heteroarylene;
R₁, R₂ and R_{T}, where present, are independently selected from an oligopeptide and R_{y}; wherein R_{y} is selected from the group consisting of hydrogen, halogen, alkyl, cycloalkyl, alkenyl, cycloalkenyl, heteroalkyl, heterocycloalkyl, acyl, aryl or heteroaryl
each R3 is independently selected from the group consisting of hydrogen, halogen, alkyl, cycloalkyl, alkenyl, cycloalkenyl, heteroalkyl, heterocycloalkyl, acyl, aryl or heteroaryl; and
n is an integer from 5 to 10,000;
or a pharmaceutically acceptable salt thereof.

10. The nanoparticle of claim 9, wherein R₁ and R₂ are both oligopeptides.

11. The nanoparticle of claim 9 or 10, wherein n is from 1 to 20.

12. The nanoparticle of any of claims 8 to 11, wherein R_{y} is independently selected from a group consisting of hydrogen, —(CH₂)ₘNH₂, —(CH₂)ₘNHMe, —(CH₂)ₘOH, —(CH₂)ₘCH₃, —(CH₂)₂(OCH₂CH₂)ₘNH₂, —(CH₂)₂(OCH₂CH₂)ₘOH or —(CH₂)₂(OCH₂CH₂)ₘCH₃ wherein m is an integer from 1 to 20.

13. The nanoparticle according to any preceding claim, further comprising an active agent.

14. A composition comprising at least one nanoparticle of any preceding claim.

15. A method for preparing nanoparticles comprising the steps of (i) preparing an end-modified poly(β-amino ester) in the presence of a sugar or sugar alcohol and (ii) preparing nanoparticles from the product of step (i).

16. The method according to claim 15, wherein the method comprises the steps of (i-a) reacting the acrylate terminated intermediate of Formula II with compounds of formulae R₁L₁H and R₂L₂H in the presence of a sugar or sugar alcohol and (ii) preparing nanoparticles from the product of step (i).

17. The method according to claim 15 or 16, wherein the sugar or sugar alcohol is present at 1 to 35 weight percent relative to the end-modified poly(β-amino ester).

18. A method for preparing nanoparticles comprising the steps of (i) preparing nanoparticles from an end-modified poly(β-amino ester) and (ii) contacting the nanoparticles with a sugar or sugar alcohol.

19. The method according to claim 18, wherein the sugar or sugar alcohol is present at 1 to 35 weight percent relative to the end-modified poly(β-amino ester).

20. A method according to any one of claims 15 to 19, wherein the sugar alcohol is mannitol.

21. A method for preparing nanoparticles comprising the steps of (i) mixing an end-modified poly(β-amino ester) with chitosan and (ii) preparing nanoparticles from the product of step (i).

22. A method according to claim 21, wherein the chitosan is present at 0.15 to 3.0 weight percent relative to the end-modified poly(β-amino ester).

23. A method according to any one of claims 15 to 22 wherein the nanoparticles are prepared in the presence of an active agent.

24. A nanoparticle produced by the method of any of claims 15 to 23.

25. A nanoparticle according to any one of claim 1 to 13 or 24 or a composition according to claim 14 for use in medicine.

26. An in vitro method of inhibiting gene expression comprising contacting one or more cells or a tissue with a nanoparticle according to any one of claim 1 to 13 or 24 or a composition according to claim 14.
